(19) United States
(12) Patent Application Publication
(10) Pub. No.: US 2025/0092383 A1
(43) Pub. Date: Mar. 20, 2025

(54) COMPOUNDS AND METHODS FOR FBXO22-MEDIATED PROTEIN DEGRADATION

(71) Applicant: Northwestern University, Evanston, IL (US)

(72) Inventors: Ananya Angela Basu, Evanston, IL (US); Xiaoyu Zhang, Evanston, IL (US)

(21) Appl. No.: 18/884,684

(22) Filed: Sep. 13, 2024

Related U.S. Application Data
(60) Provisional application No. 63/538,637, filed on Sep. 15, 2023.

(51) Int. Cl. C12N 15/10; C12N 9/10

(52) U.S. Cl. CPC C12N 15/1055; C12N 9/104; C12N 15/1082

(57) ABSTRACT

In some aspects, provided herein compounds (PROTACs) targeting the E3 ligase FBXO22 and uses thereof. In some aspects, provided herein are methods of identifying E3 ligases and compounds that support targeted protein degradation.

### PRIORITY STATEMENT

This application claims priority to U.S. Provisional Application No. 63/538,637, filed Sep. 15, 2023, the entire contents of which are incorporated herein by reference for all purposes.

### STATEMENT OF GOVERNMENT SUPPORT

This invention was made with government support under grant CA248715 awarded by The National Institutes of Health. The government has certain rights in the invention.

### SEQUENCE LISTING

The text of the computer readable sequence listing filed herewith, titled “NWEST-42376-202_SQL”, created Sep. 13, 2024, having a file size of 5,722 bytes, is hereby incorporated by reference in its entirety.

### FIELD

The disclosure relates to PROTACs targeting the E3 ligase FBXO22 and uses thereof. The disclosure further relates to methods of identifying E3 ligases and agents that support targeted protein degradation.

### BACKGROUND

Traditional small molecule drugs function by directly interfering with the activities of proteins. However, many proteins lack suitable functional sites for rational drug design, presenting challenges in targeting them with small molecules. Some proteins, such as oncogenic adaptor proteins (e.g., MYD88) or transcription factors (e.g., MYC), are even considered “undruggable.” Another complexity arises from proteins containing multiple functional domains; in such cases, compounds binding to just one domain might inadequately deactivate the protein. This is the case, for example, with IRAK4.

A promising alternative approach involves the use of small molecules that guide proteins to the cellular machinery responsible for proteolytic degradation, leading to the complete removal of the protein. This approach is called targeted protein degradation (TPD). TPD employs two types of small molecules: 1) heterobifunctional compounds, known as PROTACs (proteolysis-targeting chimeras), which link E3 ligase ligands to substrate ligands using a structurally variable linker; and 2) monofunctional compounds that form a complex involving specific E3 ligases and neo-target proteins, referred to as molecular glues (e.g., immunomodulatory drugs or IMiDs). However, only a limited subset of the 600+ human E3 ligases has been identified as supportive of targeted protein degradation. Moreover, these E3 ligases exhibit unique and restricted substrate preferences which are not currently predictable or easily controllable. Accordingly, there is a need for ligandable E3 ligases with distinct properties for use in targeted protein degradation, and a need for methods of identifying the same.

### SUMMARY

In some aspects, provided herein are proteolysis-targeting chimeras (PROTACs). In some embodiments, provided herein is a PROTAC comprising domain that binds to E3 ligase F-box protein 22 (FBXO22), a target-binding domain, and a linker connecting the domain that binds to FBXO22 to the target-binding domain.

In some embodiments, the domain that binds to FBXO22 comprises the structure:

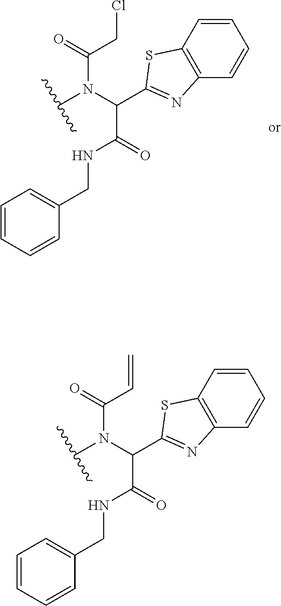 [SMILES: *N(C(=O)CCl)C(C(=O)NCc1ccccc1)c1nc2ccccc2s1 ; *N(C(=O)C=C)C(C(=O)NCc1ccccc1)c1nc2ccccc2s1]

In some embodiments, the linker comprises one or more alkylene oxide units.

In some embodiments, binding of the PROTAC to FBXO22 and to the target induces FBXO22-mediated targeted protein degradation of the target.

In some embodiments, the target is 12-kDa FK506-binding protein (FKBP12). For example, in some embodiments the target-binding domain comprises the structure:

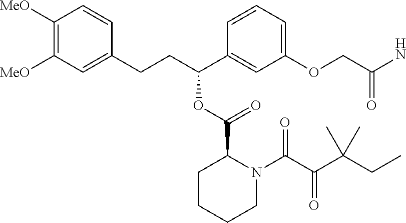

In some embodiments, the PROTAC is 22-SLF, defined by the structure:

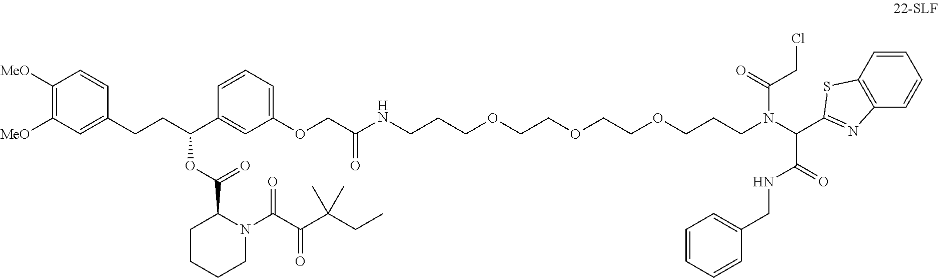 [SMILES: CCC(C)(C)C(=O)C(=O)N1CCCC[C@H]1C(=O)O[C@H](CCc1ccc(OC)c(OC)c1)c1cccc(OCC(=O)NCCCOCCOCCOCCCN(C(=O)CCl)C(C(=O)NCc2ccccc2)c2nc3ccccc3s2)c1]

In some embodiments, the PROTAC is 22-aSLF, defined by the structure:

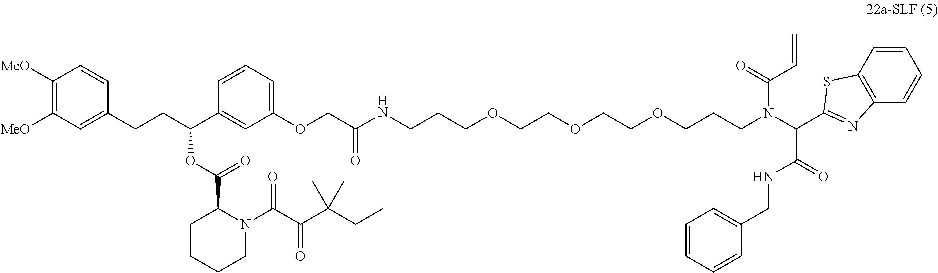 [SMILES: C=CC(=O)N(CCCOCCOCCOCCCNC(=O)COc1cccc([C@@H](CCc2ccc(OC)c(OC)c2)OC(=O)[C@@H]2CCCCN2C(=O)C(=O)C(C)(C)CC)c1)C(C(=O)NCc1ccccc1)c1nc2ccccc2s1]

In some embodiments, the target is bromodomain-containing protein 4 (BRD4). For example, in some embodiments the target-binding domain comprises the structure:

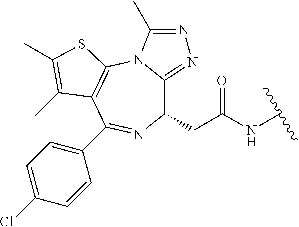 [SMILES: *NC(=O)C[C@@H]1N=C(c2ccc(Cl)cc2)c2c(sc(C)c2C)-n2c(C)nnc21]

In some embodiments, the PROTAC is 22-JQ1, defined by the structure:

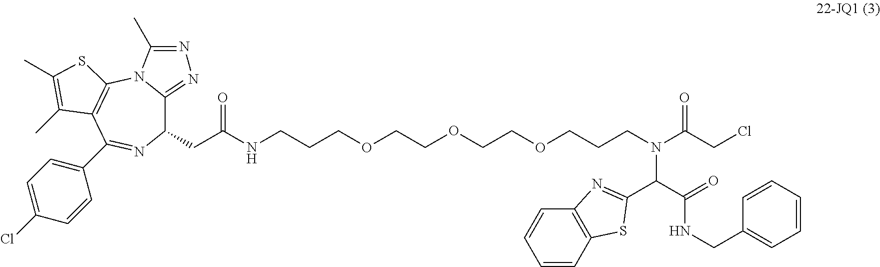 [SMILES: Cc1sc2c(c1C)C(c1ccc(Cl)cc1)=N[C@@H](CC(=O)NCCCOCCOCCOCCCN(C(=O)CCl)C(C(=O)NCc1ccccc1)c1nc3ccccc3s1)c1nnc(C)n1-2]

In some embodiments, the target is anaplastic lymphoma kinase (ALK). For example, in some embodiments the target-binding domain comprises the structure:

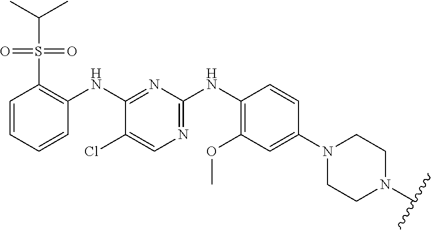 [SMILES: *N1CCN(c2ccc(Nc3ncc(Cl)c(Nc4ccccc4S(=O)(=O)C(C)C)n3)c(OC)c2)CC1]

In some embodiments, the PROTAC is 22-TAE, defined by the structure:

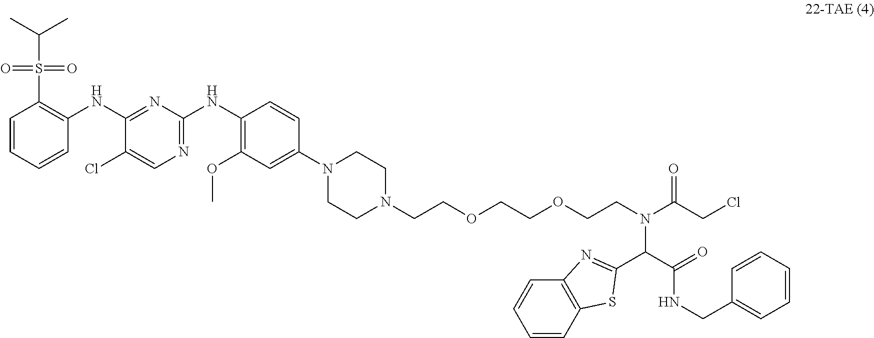 [SMILES: COc1cc(N2CCN(CCOCCOCCN(C(=O)CCl)C(C(=O)NCc3ccccc3)c3nc4ccccc4s3)CC2)ccc1Nc1ncc(Cl)c(Nc2ccccc2S(=O)(=O)C(C)C)n1]

In some aspects, provided herein are methods of promoting targeted protein degradation in a cell or a subject, or a method of treating a neurodegenerative disease or cancer in a subject, comprising administering to the cell or subject a PROTAC described herein.

In some aspects, provided herein are methods of identifying E3 ligases that support targeted protein degradation (TPD). In some embodiments, methods of identifying E3 ligases that support TPD comprise providing a sample comprising cells expressing a fusion protein and a CRISPR-Cas9 transcriptional activation system, wherein the fusion protein comprises a target protein and a reporter molecule; transducing the cells with an sgRNA library targeting human E3 ligases; treating the cells with an agent putatively directed against the target protein, wherein a reduction of signal from the reporter molecule indicates targeted protein degradation of the target protein; isolating cells with a reduced signal from the reporter molecule; and determining the expression of sgRNAs in the isolated cells relative to the expression of sgRNAs in the sgRNA library. In some embodiments, the method further comprises identifying one or more E3 ligases targeted by sgRNAs having enriched expression in the isolated cells. In some embodiments, the E3 ligases identified are determined to support targeted protein degradation of the target protein.

In some embodiments, the target protein is implicated in a disease or condition. For example, target proteins that are overexpressed in a disease or condition may be used, as degradation of the target protein may alleviate the disease or condition. As another example, target proteins involved in the pathogenesis of a disease or condition, such as those that trigger a pathogenic cell response, a pathogenic signaling pathway, etc., may be used, as targeted degradation of such a target protein may also alleviate the disease or condition. In some embodiments, the target protein is FKBP12. In some embodiments, the target protein is ALK. In some embodiments, the target protein is BRD4.

The method is not limited to use of any particular reporter protein. In some embodiments, the reporter molecule is a fluorescent protein. In some embodiments, the fusion protein comprises FKBP12-EGFP. In some embodiments, the fusion protein comprises ALK-EGFP. In some embodiments, the fusion protein comprises BRD4-EGFP.

In some embodiments, the sgRNA library comprises at least 1,000 sgRNAs. The sgRNAs target the promoter region(s) of E3 ligases. In some embodiments, the sgRNA library comprises at least 2,000 sgRNAs. In some embodiments, the sgRNA library comprises at least 3,000 sgRNAs.

In some aspects, provided herein are methods of identifying whether an agent induces targeted protein degradation (TPD) of a target protein. In some embodiments, methods of identifying whether an agent induces TPD of a target protein involve providing a sample comprising cells expressing a fusion protein and a CRISPR-Cas9 transcriptional activation system, wherein the fusion protein comprises a target protein and a reporter molecule; transducing the cells with an sgRNA library targeting human E3 ligases; treating the cells with the agent; and evaluating a signal from the reporter molecule in one or more cells, wherein a reduction of signal from the reporter molecule indicates that the agent induces TPD of the target protein.

In some embodiments, the target protein is implicated in a disease or condition. For example, target proteins that are overexpressed in a disease or condition may be used, as degradation of the target protein may alleviate the disease or condition. As another example, target proteins involved in the pathogenesis of a disease or condition, such as those that trigger a pathogenic cell response, a pathogenic signaling pathway, etc., may be used, as targeted degradation of such a target protein may also alleviate the disease or condition. For example, in some embodiments the target protein is implicated in a neurodegenerative disease, such as Parkinson's Disease or Alzheimer's disease. In some embodiments, the target protein is FKBP12. In some embodiments, the target protein is implicated in cancer. For example, in some embodiments, the target protein is ALK. As another example, in some embodiments, the target protein is BRD4.

The method is not limited to use of any particular reporter protein. In some embodiments, the reporter molecule is a fluorescent protein. In some embodiments, the fusion protein comprises FKBP12-EGFP. In some embodiments, the fusion protein comprises ALK-EGFP. In some embodiments, the fusion protein comprises BRD4-EGFP.

In some embodiments, the sgRNA library comprises at least 1,000 sgRNAs. The sgRNAs target the promoter region(s) of E3 ligases. In some embodiments, the sgRNA library comprises at least 2,000 sgRNAs. In some embodiments, the sgRNA library comprises at least 3,000 sgRNAs.

### BRIEF DESCRIPTION OF THE DRAWINGS

FIGS. 1A-1E show an E3 ligase focused CRISPR-Cas9 transcriptional activation screen identifies FBXO22 that supports 22-SLF-induced reduction in FKBP12-EGFP expression levels. FIG. 1A is a schematic representation of the steps in the CRISPR-Cas9 transcriptional activation screen. FIG. 1B shows the structure of 22-SLF. FIG. 1C is a volcano plot showing the E3 ligase focused CRISPR-Cas9 transcriptional activation screen for FKBP12-EGFP degradation after treatment of 2 μM 22-SLF in HEK293T CRISPR-Cas9 transcriptional activation cells for 24 hours. FIG. 1D shows quantitative PCR analysis of FBXO22 mRNA levels subsequent to the transduction of sgRNAs that target the promoter regions of the FBXO22 gene in HEK293T CRISPR-Cas9 transcriptional activation cells. The statistical significance was evaluated through unpaired two-tailed Student's t-tests, comparing cells transduced with FBXO22 sgRNA to control sgRNA. Statistical significance denoted ****P<0.0001. FIG. 1E shows fluorescence quantification of FKBP12-EGFP levels in HEK293T CRISPR-Cas9 transcriptional activation cells transduced with FBXO22-activating sgRNAs, subsequent to the treatment of 2 μM 22-SLF for 24 hours. The statistical significance was evaluated through unpaired two-tailed Student's t-tests, comparing cells treated with 22-SLF to DMSO. Statistical significance denoted as ***P<0.001 and ns: not significant.

FIGS. 2A-2G show that 22-SLF promotes FBXO22-dependent proteasomal degradation of FKBP12. FIG. 2A shows 22-SLF-induced FKBP12 degradation is dependent on FBXO22 and blocked by MG132 (1 μM), MLN4924 (1 μM) and SLF (25 μM) (n=3 biological independent samples). The bar graph represents quantification of the FLAG-FKBP12/HSP90 protein content. Data are presented as the mean values±s.e.m. FIG. 2B shows dose-dependent degradation of FKBP12 by 22-SLF. HEK293T cells expressing HA-FBXO22 were treated with 0.025-15 μM 22-SLF for 8 h. The graph represents quantification of the FLAG-FKBP12/HSP90 protein content. Data are presented as the mean values (n=2 biological independent samples). FIG. 2C shows time-dependent degradation of FKBP12 by 22-SLF. The bar graph represents quantification of the FLAG-FKBP12/HSP90 protein content. KO, knockout; WT, wild type. Data are presented as the mean values (n=2 biological independent samples). FIG. 2D shows 22-SLF (2 μM) induced FKBP12 degradation in A549 wild-type but not FBXO22-knockout cells (n=3 biological independent samples). The bar graph represents quantification of the FLAG-FKBP12/HSP90 protein content. Data are presented as the mean values±s.e.m. FIG. 2E shows global proteomic analysis in A549 wild-type and FBXO22-knockout cells treated with 22-SLF (2 μM) for 24 h (n=2 biological independent samples for DMSO treatment; n=3 biological independent samples for 22-SLF treatment). FIG. 2F shows the structure of 22-biotin. FIG. 2G shows the 22-biotin-conjugated streptavidin pulldown with lysates of A549 cells pretreated with 22-SLF (2, 10 or 50 μM, 2 h) followed by western blot analysis of endogenous FBXO22. The result is representative of two independent experiments with similar results. WCL, whole-cell lysate; IP, immunoprecipitate.

FIGS. 3A-3H show FBXO22 C227 and C228 are involved in 22-SLF-mediated degradation of FKBP12. FIG. 3A shows competitive cysteine-directed activity-based protein profiling (ABPP) measured the degree of blockade of IA-DTB-modified cysteines by 22-SLF. Among 5,650 quantified IA-DTB-modified peptides, four cysteines were identified on FBXO22 (C83, C117, C228, and C365). FIG. 3B shows quantification of four IA-DTB-modified peptides in FBXO22 treated with DMSO or 22-SLF (2 μM, 2 hours). FIG. 3C shows single substitution of C227 or C228 to alanine in FBXO22 partially blocked 22-SLF-induced degradation of FKBP12, while mutating both C227 and C228 to alanine in FBXO22 completely abolished the degradation. FIG. 3D shows global proteomic analysis in HEK293T cells expressing HA-FBXO22 wildtype or C227AC228A double mutant treated with 22-SLF (0.5 μM, 24 hours). Data are presented as the mean values (n=2 biological independent samples for DMSO treatment; n=3 biological independent samples for 22-SLF Treatment). FIG. 3E shows modeling study revealing several hydrogen bond interactions between the electrophilic portion of 22-SLF and a pocket in FBXO22 involving C227 and C228. FIG. 3F shows sequence alignment of human FBXO22 (aa 219-230) and mouse FBXO22 (aa 218-229). FIG. 3G shows mouse FBXO22 supporting 22-SLF-induced FKBP12 degradation. The bag graph represents quantification of the FLAG-FKBP12/HSP90 protein content. Data are presented as mean values (n=2 biological independent samples). FIG. 3H shows substitution of both C226 and C227 in mouse FBXO22 abolished 22-SLF-induced degradation of FKBP12. The bar graph represents quantification of the FLAG-FKBP12/HSP90 protein content. Data are presented as the mean values (n=2 biological independent samples).

FIGS. 4A-4C show 22-SLF induces the formation of a ternary complex involving 22-SLF, FKBP12 and FBXO22. FIG. 4A shows results of a coimmunoprecipitation assay revealing that HA-FBXO22 wild type, but not HA-FBXO22 C227A/C228A double mutant, coimmunoprecipitated with FLAG-FKBP12 in the presence of 22-SLF and MG132. The bar graph represents quantification of the immunoprecipitated HA-FBXO22 protein content compared to the HA-FBXO22 protein in whole cell lysates (WCL). Data are presented as the mean values (n=2 biological independent samples). FIG. 4B shows FKBP12-directed enrichment proteomic analysis revealed that HA-FBXO22 wild type and its associated components in the SKP1-CUL1-RBX1 E3 complex coimmunoprecipitated with FLAG-FKBP12 in the presence of 22-SLF (2 μM) and MG132 (5 μM). FIG. 4C shows FKBP12-directed enrichment proteomic analysis revealed that HA-FBXO22 C227A/C228A double mutant and its associated components in the SKP1-CUL1-RBX1 E3 complex did not coimmunoprecipitate with FLAG-FKBP12 in the presence of 22-SLF (2 μM) and MG132 (5 μM). Data are presented as the mean values (n=2 biological independent samples).

FIGS. 5A-5E show harnessing FBXO22 for the degradation of BRD4 and EML4-ALK. FIG. 5A shows the structure of 22-JQ1. FIG. 5B shows 22-JQ1 (1 and 2 μM, 24 h) induced BRD4 degradation in A549 wild-type but not FBXO22-knockout cells. The bar graph represents quantification of the BRD4/β-actin protein content. Data are presented as the mean values (n=2 biological independent samples). FIG. 5C shows global proteomic analysis in A549 wild-type and FBXO22-knockout cells treated with 2 μM 22-JQ1 for 24 h. Data are presented as the mean values (n=2 biological independent samples). FIG. 5D shows the structure of 22-TAE. FIG. 5E shows 22-TAE (1 and 2 μM, 24 h) induced EML4-ALK degradation in H2228 wild-type but not FBXO22-knockout cells. The bar graph represents quantification of the EML4-ALK/β-actin protein content. Data are presented as the mean values (n=2 biological independent samples).

FIGS. 6A-6E show generation of CRISPR-Cas9 transcriptional activation cells for the discovery of E3 ligases supporting the degradation of FKBP12-EGFP. FIG. 6A shows the construct of FKBP12-EGFP and a schematic representation of the generation of FKBP12-EGFP expressing cells. FIG. 6B shows structures of Len-SLF and SLF. FIG. 6C shows fluorescence quantification of FKBP12-EGFP levels in HEK293T cells treated with 2 μM of Len-SLF or 20 μM of SLF for 24 hours. The statistical significance was evaluated through unpaired two-tailed Student's t-tests, comparing cells treated with Len-SLF or SLF to DMSO. Statistical significance denoted as ***P<0.001 and ns: not significant. FIG. 6D shows the constructs used for the CRISPR-Cas9 transcriptional activation screen. FIG. 6E shows quantitative PCR analysis of IL-1β mRNA levels subsequent to the transduction of sgRNAs that target the promoter regions of the IL-1β gene in HEK293T CRISPR-Cas9 transcriptional activation cells. The statistical significance was evaluated through unpaired two-tailed Student's t-tests, comparing cells transduced with IL-1β sgRNA to control sgRNA. Statistical significance denoted as ****P<0.0001.

FIG. 7 shows the procedure of fluorescence-activated cell sorting for the CRISPR-Cas9 transcriptional activation screen.

FIGS. 8A-8E show that 22-SLF promotes FBXO22-dependent proteasomal degradation of FKBP12. FIG. 8A shows tumor/normal ratio values of gene expression of FBXO22 and CRBN. Data is obtained from Gene Expression Profiling Interactive Analysis (GEPIA). FIG. 8B shows genomic PCR confirms FBXO22 knockout in HEK293T, A549, MDA-MB-231 and PC3 cells. FIG. 8C shows global proteomic analysis confirms FBXO22 knockout in HEK293T, A549, MDA-MB-231 and PC3 cells. KDM4A and KDM4B are two reported substrates of FBXO22. FIG. 8D shows 22-SLF promoted reduction in FKBP12 levels in MDA-MB-231 and PC3 wildtype, but not FBXO22 KO cells. FIG. 8E shows global proteomic analysis in A549 wildtype and FBXO22 knockout cells treated with 22-SLF (2 μM, 24 hours).

FIGS. 9A-9B show FBXO22 C227 and C228 are involved in 22-SLF-mediated degradation of FKBP12. FIG. 9A shows interactome studies of FBXO22 wildtype and C227AC228A double mutant revealed that FBXO22 C227AC228A double mutant was assembled into the SKP1-CUL1-RBX1 E3 complex. FIG. 9B shows global proteomic analysis in HEK293T cells expressing HA-FBXO22 wildtype or C227AC228A double mutant treated with 22-SLF (0.5 μM, 24 hours).

FIG. 10 shows sequence alignment of human and mouse FBXO22. Sequence alignment was performed in Clustal Omega.

FIG. 11A-11B show the acrylamide variant of 22-SLF, 22a-SLF, induced the degradation of FKBP12. FIG. 11A shows the structure of 22a-SLF. FIG. 11B shows a comparison of FKBP12 degradation by 22-SLF and 22a-SLF. HEK293T cells expressing HA-FBXO22 were treated with 0.5, 1, or 2 μM of 22-SLF or 22a-SLF for 8 hours. The graph represents quantification of the FLAG-FKBP12/HSP90 protein content. Data are presented as mean values (n=2 biological independent samples).

### DETAILED DESCRIPTION

### 1. Definitions

Although any methods and materials similar or equivalent to those described herein can be used in the practice or testing of embodiments described herein, some preferred methods, compositions, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to ±10% of the indicated number. For example, “about 10%” may indicate a range of 9% to 11%, and “about 1” may mean from 0.9-1.1. Other meanings of “about” may be apparent from the context, such as rounding off; for example, “about 1” may also mean from 0.5 to 1.4.

As used herein, the terms “comprise”, “include”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence of the present disclosure after aligning the two sequences and introducing gaps, if necessary, to achieve the maximum percent identity. Hence, in case a nucleic acid or protein is longer than a reference sequence, additional nucleotides or amino acids that do not align with the reference sequence are not taken into account for determining sequence identity. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof).

An “effective amount” refers to an amount sufficient to elicit a desired biological response (e.g., treating a condition). As will be appreciated by those skilled in the art, the effective amount may vary depending on such factors as the desired biological endpoint, the pharmacokinetics, the condition being treated, the mode of administration, and the age and health of the subject. An effective amount encompasses therapeutic and prophylactic treatment. For example, a “therapeutically effective amount” is an amount sufficient to provide a therapeutic benefit in the treatment of a condition, or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

### 2. Proteolysis Targeting Chimeras

In some aspects, provided herein are compounds. In some embodiments, the compounds are proteolysis-targeting chimeras, or PROTACs. As used herein, the term “proteolysis-targeting chimera” or “PROTAC” are used interchangeably and refer to a heterobifunctional molecule comprising three components: a target-binding domain (also referred to as a “warhead”), a linker, and an E3 ubiquitin ligase binding domain (also referred to as an “anchor”). The linker connects the target-binding domain to the E3 ubiquitin ligase binding domain. PROTACs achieve degradation of a target protein by simultaneous binding of the target-binding domain to the target protein and the E3 ubiquitin ligase binding domain to an E3 ubiquitin ligase, resulting in ubiquitylation of the target protein and subsequent degradation thereof by the proteasome. This process is referred to as “targeted protein degradation” or “TPD”.

E3 ubiquitin ligases, also referred to herein as E3 ligases or ubiquitin ligases, refer to a family of proteins that enable movement of ubiquitin from a ubiquitin carrier (e.g. an E2 ubiquitin-conjugating enzyme) to a substrate (e.g. a protein substrate). E3 ligases are involved in many processes including protein degradation, DNA repair, cell cycle progression, transcriptional regulation, signal transduction, and the like. In some embodiments, the E3 ligase is F-box protein 22 (FBXO2 or FBX22), identified herein as an E3 ligase supportive of targeted protein degradation.

In some embodiments, provided herein is a proteolysis-targeting chimera (PROTAC) comprising:
a) domain that binds to E3 ligase F-box protein 22 (FBXO22),
b) a target-binding domain, and
c) a linker connecting the domain that binds to FBXO22 to the target-binding domain.

FBXO22 refers to a member of the F-box protein family which is characterized by an approximately 40 amino acid referred to as the F-box which functions in phosphorylation-dependent ubiquitination. In some embodiments, FBXO22 refers to a protein comprising an amino acid sequence having at least 80% sequence identity to:
MEPVGCCGECRGSSVDPRSTFVLSNLAEVVERVLTFLPAKALLRVACVCRLWRE CVRRVLRTHRSVTWISAGLAEAGHLEGHCLVRVVAEELENVRILPHTVLYMADSETFIS LEECRGHKRARKRTSMETALALEKLFPKQCQVLGIVTPGIVVTPMGSGSNRPQEIEIGES GFALLFPQIEGIKIQPFHFIKDPKNLTLERHQLTEVGLLDNPELRVVLVFGYNCCKVGASN YLQQVVSTFSDMNIILAGGQVDNLSSLTSEKNPLDIDASGVVGLSFSGHRIQSATVLLNE DVSDEKTAEAAMQRLKAANIPEHNTIGFMFACVGRGFQYYRAKGNVEADAFRKFFPSV PLFGFFGNGEIGCDRIVTGNFILRKCNEVKDDDLFHSYTTIMALIHLGSSK (SEQ ID NO: 1).

In some embodiments, the domain that binds to FBXO22 comprises the structure:

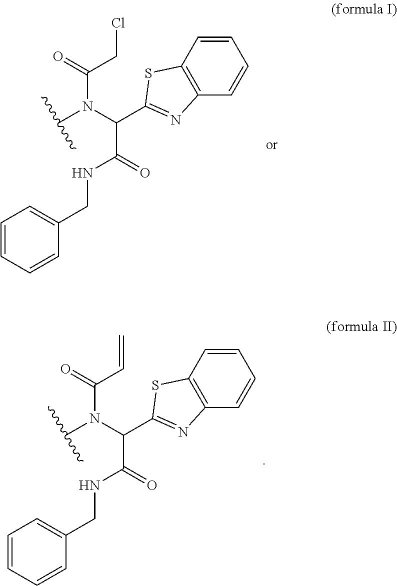 [SMILES: *N(C(=O)CCl)C(C(=O)NCc1ccccc1)c1nc2ccccc2s1 ; *N(C(=O)C=C)C(C(=O)NCc1ccccc1)c1nc2ccccc2s1]

As used herein, in chemical structures the indication:

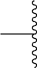

represents a point of attachment of one moiety to another moiety. For example, the indication may represent a point of attachment of the domain that binds to FBXO22 (e.g. the structure of formula I or formula II), to the linker (e.g. the linker that connects the domain that binds to FBXO22 to the target-binding domain). As another example, the indication may represent a point of attachment of the target-binding domain to the linker (e.g. the linker that connects the target-binding domain to the domain that binds to FBXO22).

Any suitable linker may be used. In some embodiments, the linker comprises a combination of one or more chemical motifs (e.g. units or atoms). Exemplary linker motifs (units) include, for example, PEG, alkyl, glycol, alkyne, triazole, piperazine, and piperidine motifs. In some embodiments, the linker comprises a single type of chemical motif (e.g. unit). In some embodiments, the linker comprises multiple different types of chemical motifs (e.g. units). In some embodiments, the linker comprises one or more alkylene oxide units. For example, in some embodiments, the linker comprises one or more ethylene oxide units. Generally speaking, the linker should be of a suitable length to achieve a sufficient distance between the protein-binding domain (e.g. the warhead) and the FBXO22 binding domain (e.g. the anchor) to avoid steric repulsions when the target protein and FBXO22 are both bound to the PROTAC, while not being too long such that the PROTAC fails to facilitate ubiquitylation and degradation of the target. In some embodiments, the linker comprises 3 to 50 units. In some embodiments, the linker comprises 3 to 40 units, 3 to 30 units, 3 to 25 units, 3 to 20 units, 3 to 15 units, or 3 to 10 units. In some embodiments, the linker is a flexible linker.

In some embodiments, the linker comprises one or more alkylene glycol repeat units, such as ethylene glycol or propylene glycol repeat units. For example, in some embodiments, the linker comprises a poly- or oligo-ethylene glycol chain:

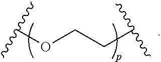

In some embodiments, p is 1 to 30. In some embodiments, p is 1 to 20. In some embodiments, p is 1 to 10. In some embodiments, p is 1 to 6. For example, in some embodiments p is 1, 2, 3, 4, 5, or 6.

In some embodiments, the linker comprises comprises a group of formula:

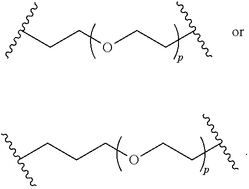

In some embodiments, p is 1 to 30. In some embodiments, p is 1 to 20. In some embodiments, p is 1 to 10. In some embodiments, p is 1 to 6. For example, in some embodiments, p is 1, 2, 3, 4, 5, or 6.

The target-binding domain may bind to any suitable target protein for which targeted protein degradation (TPD) is desired. In some embodiments, the target protein is 12-kDa FK506-binding protein (FKBP12). FKBP12 is a member of the FK506 binding protein (FKBP) family. In some embodiments, the target protein is human FKBP12.

In some embodiments, the target protein is FKBP12 and the target-binding domain comprises the structure:

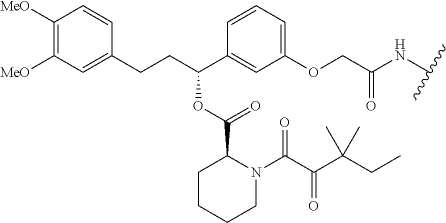 [SMILES: *NC(=O)COc1cccc([C@@H](CCc2ccc(OC)c(OC)c2)OC(=O)[C@@H]2CCCCN2C(=O)C(=O)C(C)(C)CC)c1]

In some embodiments, the PROTAC is 22-SLF. 22-SLF is defined by the structure:

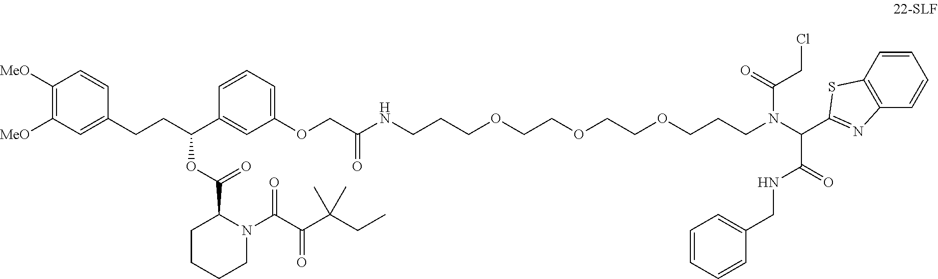 [SMILES: CCC(C)(C)C(=O)C(=O)N1CCCC[C@H]1C(=O)O[C@H](CCc1ccc(OC)c(OC)c1)c1cccc(OCC(=O)NCCCOCCOCCOCCCN(C(=O)CCl)C(C(=O)NCc2ccccc2)c2nc3ccccc3s2)c1]

In some embodiments, the PROTAC is 22a-SLF. 22a-SLF is an acrylamide variant of 22-SLF and has the following structure:

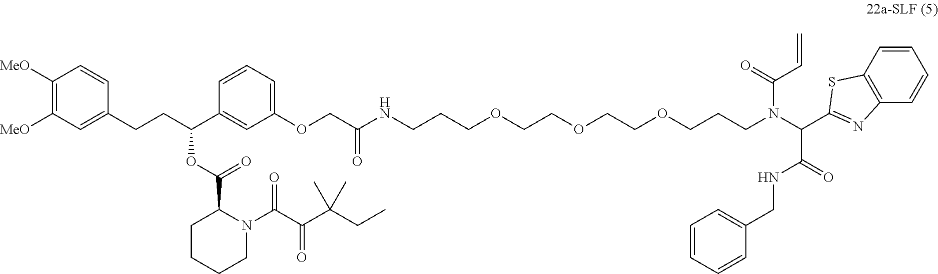 [SMILES: C=CC(=O)N(CCCOCCOCCOCCCNC(=O)COc1cccc([C@@H](CCc2ccc(OC)c(OC)c2)OC(=O)[C@@H]2CCCCN2C(=O)C(=O)C(C)(C)CC)c1)C(C(=O)NCc1ccccc1)c1nc2ccccc2s1]

In some embodiments, the target protein is Bromodomain-containing protein 4 (BRD4). BRD4 is a member of the bromodomain and extraterminal domain (BET) family which also includes BRD2, BRD3, and BRDT. BRD4 contains two bromodomains that recognize acetylated lysine residues. BRD4 is a transcriptional and epigenetic regulator and has been implicated in embryogenesis, cell regulation, and cancer development.

In some embodiments, the target protein is BRD4 and the target-binding domain comprises the following structure:

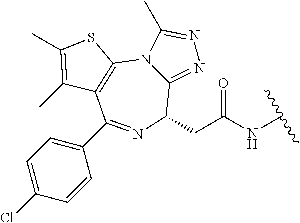 [SMILES: *NC(=O)C[C@@H]1N=C(c2ccc(Cl)cc2)c2c(sc(C)c2C)-n2c(C)nnc21]

In some embodiments, the PROTAC is 22-JQ1. 22-JQ1 is defined by the following structure:

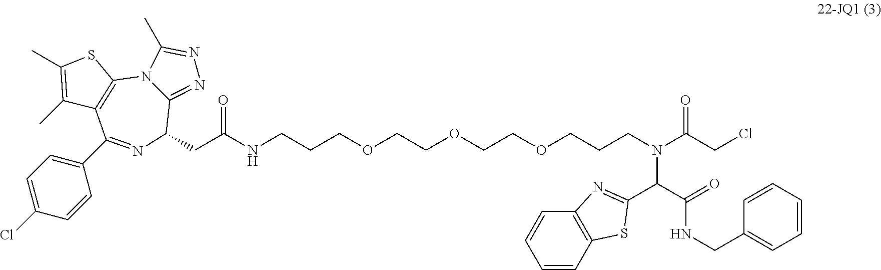 [SMILES: Cc1sc2c(c1C)C(c1ccc(Cl)cc1)=N[C@@H](CC(=O)NCCCOCCOCCOCCCN(C(=O)CCl)C(C(=O)NCc1ccccc1)c1nc3ccccc3s1)c1nnc(C)n1-2]

In some embodiments, the target protein is anaplastic lymphoma kinase (ALK). ALK is a receptor tyrosine kinase involved in regulation of cellular growth and differentiation. ALK is implicated in multiple cancer types, including lymphoma and non-small cell lung cancer.

In some embodiments, the target protein is ALK and the target-binding domain comprises the following structure:

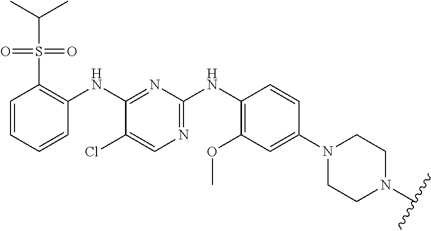 [SMILES: *N1CCN(c2ccc(Nc3ncc(Cl)c(Nc4ccccc4S(=O)(=O)C(C)C)n3)c(OC)c2)CC1]

In some embodiments, the PROTAC is 22-TAE. 22-TAE is defined by the following structure:

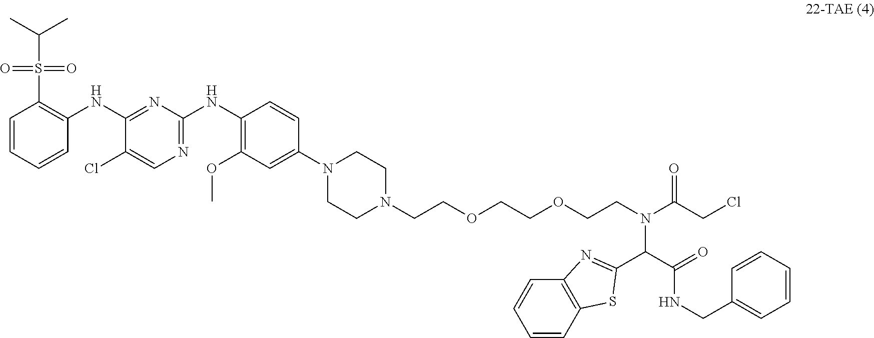 [SMILES: COc1cc(N2CCN(CCOCCOCCN(C(=O)CCl)C(C(=O)NCc3ccccc3)c3nc4ccccc4s3)CC2)ccc1Nc1ncc(Cl)c(Nc2ccccc2S(=O)(=O)C(C)C)n1]

As evidenced by the multiple exemplary PROTACs described above and in the accompanying Examples, provided herein is a PROTAC platform that can be designed to target any desired target protein to facilitate targeted protein degradation thereof. In the accompanying Examples, it is demonstrated that PROTACs comprising the FBXO22 binding domain of formula I or formula II bind effectively to FBXO22 and to multiple different target proteins, thereby forming complexes supportive of targeted protein degradation of a variety of proteins. As such, the target-binding domain is not limited to the exemplary target-binding domain structures provided above, rather the domain that binds to FBXO22 (e.g. the structure of formula I or the structure of formula II) can be attached (via the linker) to any suitable target-binding domain. For example, the target protein may be implicated in a disease or condition. For example, the target protein may be overexpressed in a disease or condition, and targeted protein degradation of the target protein may be desired to reduce the levels of the target and thereby treat the disease or condition. In some embodiments, the target protein is implicated in a neurological disease or condition, such as Alzheimer's disease or Parkinson's disease. In some embodiments, the target protein is implicated in cancer. In some embodiments, the target protein lacks functional sites for other forms of therapeutics (e.g. small molecules, antibodies, etc.).

### 3. Methods of Use

In some embodiments, the PROTACS herein provided herein find use in methods of promoting targeted protein degradation in a cell or in a subject. In some embodiments, provided herein is a method of promoting targeted protein degradation in a cell or a subject, the method comprising providing to the cell or subject a PROTAC described herein. The target protein may be implicated in a disease or condition, and as such TPD of the target protein may treat the disease or condition. As such, in some embodiments provided herein are methods of promoting TPD in a subject afflicted with a particular disease or condition for which TPD of the target protein is beneficial.

In some embodiments, the target protein is FKBP12, BRD4, or ALK. In some embodiments, the PROTAC is 22-SLF, 22-aSLF, 22-JQ1, or 22-TAE. As described above, these exemplary PROTACs are not to be construed as limiting and any suitable PROTAC comprising the FBXO22 binding domain of formula I or formula I attached to a target-binding domain via a linker can be designed and implemented in methods of promoting targeted degradation of a desired target protein.

In some embodiments, provided herein is a method of promoting targeted protein degradation of FKBP12 in a cell or a subject. In some embodiments, comprising providing to the cell or subject 22-SLF or 22a-SLF. FKBPs are implicated in some neurodegenerative diseases, such as Alzheimer's disease (AD) and Parkinson's disease (PD). In some embodiments, the subject has a neurodegenerative disease, such as AD or PD. In some embodiments, provided herein is a method of promoting TPD of FKBP12 in a subject having or suspected of having a neurodegenerative disease, comprising providing to the subject a PROTAC provided herein targeting FKBP12 (e.g. 22-SLF or 22-aSLF). In some embodiments provided herein are methods of treating a neurodegenerative disease in subject, comprising providing to the subject a composition comprising 22-SLF or 22-aSLF.

In some embodiments, provided herein is a method of promoting targeted protein degradation of BRD4 in a cell or a subject, comprising providing to the cell or subject 22-JQ1. BRD4 is a regulator of cancer cell proliferation. In some embodiments, the subject has cancer. In some embodiments, provided herein is a method of promoting TPD of BRD4 in a subject having or suspected of having cancer, comprising providing to the subject a PROTAC provided herein targeting BRD4 (e.g. 22-JQ1). In some embodiments provided herein are methods of treating cancer in a subject, comprising providing to the subject 22-JQ1.

In some embodiments, provided herein is a method of promoting targeted protein degradation of ALK in a cell or a subject. ALK is implicated in various cancers. In some embodiments, the subject has cancer. In some embodiments, provided herein is a method of promoting TPD in a subject having or suspected of having cancer, comprising providing to the subject a PROTAC provided herein targeting ALK (e.g. 22-TAE). In some embodiments provided herein is a method of treating cancer in a subject, comprising providing to the subject 22-TAE.

The PROTAC may be provided to the subject by any suitable administration route. Suitable administration routes include, for example, oral administration and parenteral administration (e.g. by injection, such as intravenous, intradermal, subcutaneous, intramuscular, etc.).

In some embodiments, the PROTAC is comprised in a composition (e.g. a pharmaceutical composition) comprising a pharmaceutically acceptable carrier or excipient. Reference to providing or administering the PROTAC to the subject is inclusive of providing or administering a composition (e.g. a pharmaceutical composition) comprising the PROTAC to the subject. The phrase “pharmaceutically acceptable,” as used in connection with compositions of the present disclosure, refers to molecular entities and other ingredients of such compositions that are physiologically tolerable and do not typically produce undesirable reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. The pharmaceutically acceptable carrier should also be compatible with the active ingredient of the composition (e.g., the PROTAC). Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The dose of the PROTAC provided to the subject may depend on the severity of the condition, the individual patient parameters including age, physical condition, size, gender and weight, the duration of the treatment, the nature of concurrent therapy (if any), the specific route of administration, the precise PROTAC used, and like factors within the knowledge and expertise of the health practitioner. In some embodiments, the effective amount is sufficient to treat a disease or condition (e.g. Alzheimer's Disease, Parkinson's Disease, cancer) in the subject. For example, in some embodiments the effective amount alleviates, relieves, ameliorates, improves, reduces the symptoms, or delays the progression of the disease or condition in the subject. For example, in some embodiments the effective amount induces a TPD of a protein implicated in the disease or condition, thereby treating the disease or condition in the subject.

It will be appreciated that appropriate dosages can vary from patient to patient. Determining the optimal dosage will generally involve the balancing of the level of therapeutic benefit against any risk or deleterious side effects of the treatments of the present disclosure. The amount and route of administration will ultimately be at the discretion of the physician, although generally the dosage will be to achieve local concentrations at the site of action which achieve the desired effect without causing substantial harmful or deleterious side-effects.

The PROTAC may be provided to the subject in be in a single dose or in multiple doses throughout the course of treatment. Methods of determining the most effective means and dosage of administration are well known to those of skill in the art and will vary with the exact PROTAC and route of administration used for therapy, the severity of the disease or condition, and the subject being treated. Single or multiple administrations can be carried out with the dose level and pattern being selected by the treating physician.

In some embodiments, a given dose is provided to the subject continuously or intermittently over the course of a suitable dosing window. For example, the dosing window may be 10 minutes to 6 hours, 20 minutes to 5 hours, 30 minutes to 4 hours, or about 1 to 3 hours. In some embodiments, the PROTAC is provided to the subject once per day. In some embodiments, the PROTAC is provided to the subject multiple times per day. In some embodiments, the PROTAC is provided to the subject every other day, every 3 days, every 4 days, every 5 days, every 6 days, every 7 days, every 8 days, every 9 days, every 10 days, every 2 weeks, every 3 weeks, monthly, every 2 months, every 3 months, every 4 months, every 5 months, every 6 months, annually, etc. The PROTAC may be administered until a desired reduction of symptoms is achieved.

In some embodiments, the PROTAC is provided to the subject in combination with other therapies for the disease or condition. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

### 4. Methods of Identifying E3 Ligases Supportive of Targeted Protein Degradation

Targeted protein degradation offers several advantages. It can transform inert protein-binding small molecules into functional protein degraders, therefore expanding the range of druggable proteins in human proteome. Additionally, this approach can function catalytically, potentially reducing the required drug concentrations for a therapeutic impact. For example, several anticancer drugs in the market or under clinical evaluation operate through this mechanism. Despite these successful cases, only a limited subset of the 600+ human E3 ligases has been identified as supportive of targeted protein degradation.

Ubiquitin ligases (E3 ligases) are proteins that transfer ubiquitin from a ubiquitin carrier (e.g. an E2 ubiquitin-conjugating enzyme) to a protein substrate. Commonly, E3 ligases ubiquitinate the substrate with Lys-48 linked chains of ubiquitin, which targets the protein for degradation by the proteasome. However, many other types of linkages are possible and alter a protein's activity, interactions, or localization. Ubiquitination by E3 ligases regulates diverse areas such as cell trafficking, protein degradation, DNA repair, and cell signaling.

To comprehensively evaluate the candidate PROTAC's potential to engage various human E3 ligases, a novel strategy based on CRISPR transcriptional activation screening was developed herein. This method can be implemented within a single cell line and has the potential to assess the interaction capabilities a multitude of human E3 ligases with candidate PROTACs, thus facilitating targeted protein degradation. This strategy involves the development of a focused single guide RNA (sgRNA) library, which is tailored to target the promoter regions of human E3 ligase genes. By employing this library, the expression of human E3 ligases is effectively induced within a specific cell line. This systematic approach provides a means to identify previously undiscovered E3 ligases suitable for targeted protein degradation. Through the utilization of this technique, an E3 ligase F-box protein 22 (FBXO22) was identified herein, which actively facilitated the degradation of FKBP12 by an electrophilic bifunctional compound designed to bind FKBP12. The application of the FBXO22-targeting ligand provided additional PROTACs capable of degrading multiple endogenous proteins. This innovative strategy not only expands the repertoire of E3 ligases available for targeted protein degradation but also demonstrates the utility of CRISPR transcriptional activation screens in efficiently assessing candidate PROTAC interactions with a diverse range of human E3 ligases.

In some aspects, provided herein is a method of identifying E3 ligases that support targeted protein degradation (TPD). In some embodiments, an E3 ligase that “supports” targeted protein degradation refers to an E3 ligase that can be targeted/bound by heterobifunctional compounds, known as PROTACs (proteolysis-targeting chimeras), which link E3 ligase ligands to substrate ligands. In some embodiments, the method comprises providing a sample comprising cells expressing a fusion protein and a CRISPR-Cas9 transcriptional activation system, wherein the fusion protein comprises a target protein and a reporter molecule; transducing the cells with an sgRNA library targeting human E3 ligases; and treating the cells with an agent putatively directed against the target protein. In some embodiments, the agent putatively directed against the target protein is a putative heterobifunctional molecule or compound that links an E3 ligase to the target protein, thus facilitating targeted degradation of the target protein. A reduction of signal from the reporter molecule indicates targeted protein degradation of the target protein. In some embodiments, the method further comprises isolating cells with a reduced signal from the reporter molecule; and determining the expression of sgRNAs in the isolated cells relative to the expression of sgRNAs in the sgRNA library. In some embodiments, the method further comprises identifying one or more E3 ligases targeted by sgRNAs having enriched expression in the isolated cells, wherein the E3 ligases identified are determined to support targeted protein degradation of the target protein. For example, the sequence of the enriched sgRNAs can be identified and used to determine which E3 ligase is targeted by a given sgRNA.

In some aspects, provided herein are methods of identifying compounds that induce TPD of a target protein. In some embodiments, provided herein is a method of identifying agents that induce targeted protein degradation (TPD) of a target protein, the method comprising: providing a sample comprising cells expressing a fusion protein and a CRISPR-Cas9 transcriptional activation system, wherein the fusion protein comprises a target protein and a reporter molecule; transducing the cells with an sgRNA library targeting human E3 ligases; treating the cells with the agent; and evaluating a signal from the reporter molecule in one or more cells, wherein a reduction of signal from the reporter molecule indicates that the agent induces TPD of the target protein. In some embodiments, the candidate compound is a putative heterobifunctional molecule or compound that links an E3 ligase to the target protein, thus facilitating targeted degradation of the target protein.

For any of the methods described herein, any suitable target protein may be used. In some embodiments, the target protein is a protein implicated in a disease or condition, such that degradation of the protein would have a beneficial therapeutic effect. In some embodiments, the target protein is an FK506 binding protein, also referred to as an FK506-binding protein (FKBP). FKBPs are a large family of proteins that possess peptidyl prolyl cis/trans isomerase (PPIase) domains. In some embodiments, the target protein is FKBP12. In some embodiments, the target protein is anaplastic lymphoma kinase (ALK). The anaplastic lymphoma kinase (ALK) gene plays a crucial role in driving the progression of a subset of non-small-cell lung cancers. Accordingly, FBXO22-mediated degradation of ALK may be used for the treatment of lung cancer. PROTACS targeting FBXO22 can be identified using the screening methods provided herein and PROTACS shown to successfully initiate FBXO22-mediated degradation of ALK can be used for the treatment of lung cancer. In some embodiments, the target protein is BRD4.

In some embodiments, the reporter molecule is a fluorescent protein. Any suitable fluorescent protein may be used, including green fluorescent proteins, yellow fluorescent proteins, red fluorescent proteins, cyan fluorescent proteins, etc., including derivatives thereof. In some embodiments, the fusion protein comprises FKBG12-EGFP, ALK-EGFP, or BRD4-EGFP.

In some embodiments, the sgRNA library comprises at least 1,000 sgRNAs. In some embodiments, the sgRNA library comprises at least 2,000 sgRNAs. In some embodiments, the sgRNA library comprises at least 3,000 sgRNAs. The sgRNA library contains a plurality of sgRNAs that target the promoter regions of human E3 ligase genes.

In some embodiments, the CRISPR-Cas9 transcriptional activation system comprises at least a catalytically inactive Cas9 enzyme linked to a transcriptional activator. In some embodiments, the CRISPR-Cas9 transcriptional activation system further comprises one or more coactivators. In some embodiments, the transcriptional activator is VP64. In some embodiments, the coactivators comprise one or more of p65 and HSF1. In some embodiments, the CRISPR-Cas9 transcriptional activation system comprises dCas9-VP64 and MS2-P65-HSF1.

Any suitable cell line may be used in the methods described herein. In some embodiments, the cells comprise HEK cells. In some embodiments, the cells comprise HEK293T cells. However, the disclosure is not limited to any particular cell type.

As shown in the accompanying Examples, the methods described herein were used to identify the E3 ligase FBXO22 as a ligase supportive of targeted protein degradation.

### EXAMPLES

### Example 1

A Focused CRISPR-Cas9 Transcriptional Activation Screen Identifies an E3 Ligase FBXO22 that Supports Electrophilic PROTAC-Induced Degradation of FKBP12.

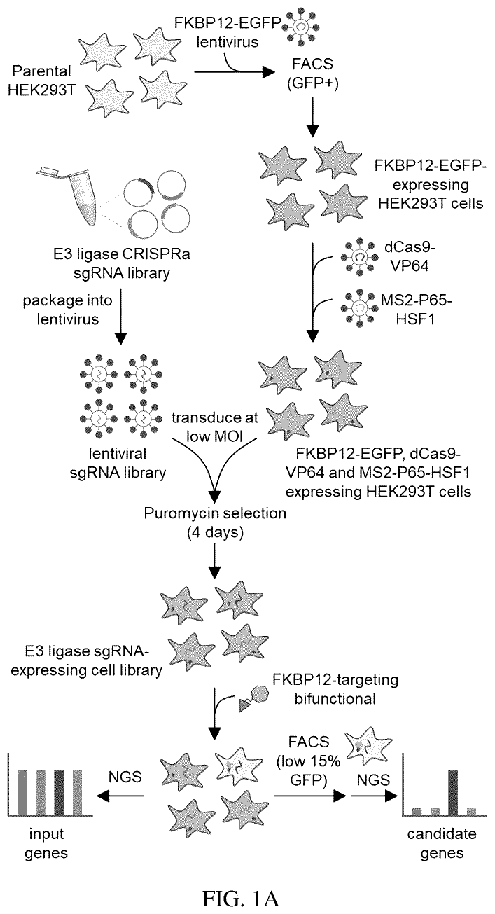

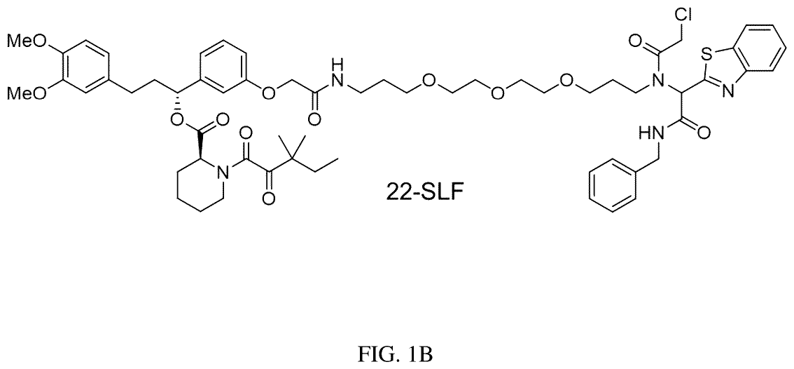

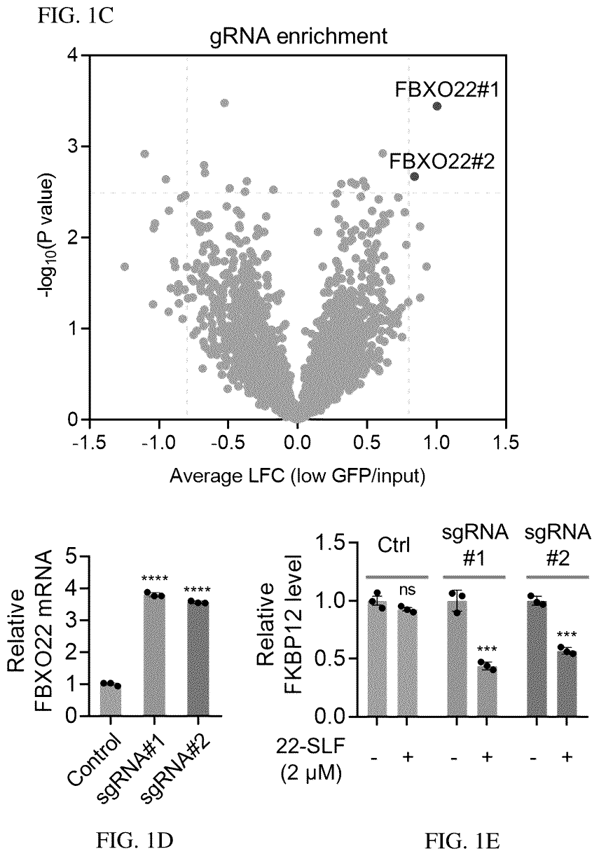

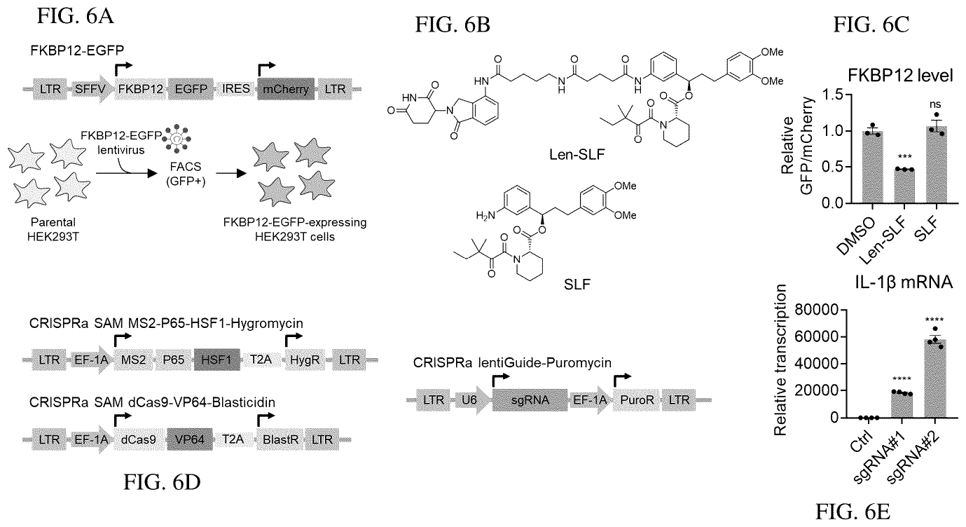

FKBP12 was used as the target protein for the degradation study. FKBP12 is a cytosolic prolyl isomerase used to investigate ligand-induced protein degradation. To establish CRISPR-Cas9 transcriptional activation cells for the exploration of FKBP12 degraders, HEK293T cells were initially transduced with lentivirus carrying FKBP12-EGFP and subsequently GFP+ cells were sorted (FIG. 1a and FIG. 6a). The incorporation of a fluorescent tag allowed fluorescent intensity to be used as a measurable parameter to assess FKBP12 protein levels. The degradation of FKBP12-EGFP was first confirmed using Len-SLF (FIG. 6b,c), a PROTAC targeting FKBP12 through recruiting CRBN. In contrast, the FKBP12 binder alone, SLF (synthetic ligand of FKBP), did not induce degradation of FKBP12-EGFP (FIG. 6b,c). This data indicates the feasibility of using FKBP12-EGFP fusion protein to investigate FKBP12 targeting degraders. Subsequently, in FKBP12-EGFP-expressing cells, both dCas9-VP64 and MS2-P65-HSF1 were stably expressed (FIG. 1a and FIG. 6d). This second-generation CRISPR-Cas9 transcriptional activation system incorporates the transcriptional activator VP64 along with additional coactivators p65 and HSF1, yielding enhanced transcriptional activation capabilities across diverse genes. To validate the efficacy of gene transcriptional activation, IL-1β was selected and two constructs with single-guide RNA (sgRNA) sequences targeting IL-1β promoter regions were developed. Transduction of FKBP12-EGFP, dCas9-VP64, and MS2-P65-HSF1-expressing cells with both IL-1β sgRNAs resulted in substantial elevation of IL-1 gene expression (FIG. 6e), confirming the effectiveness of this cell system.

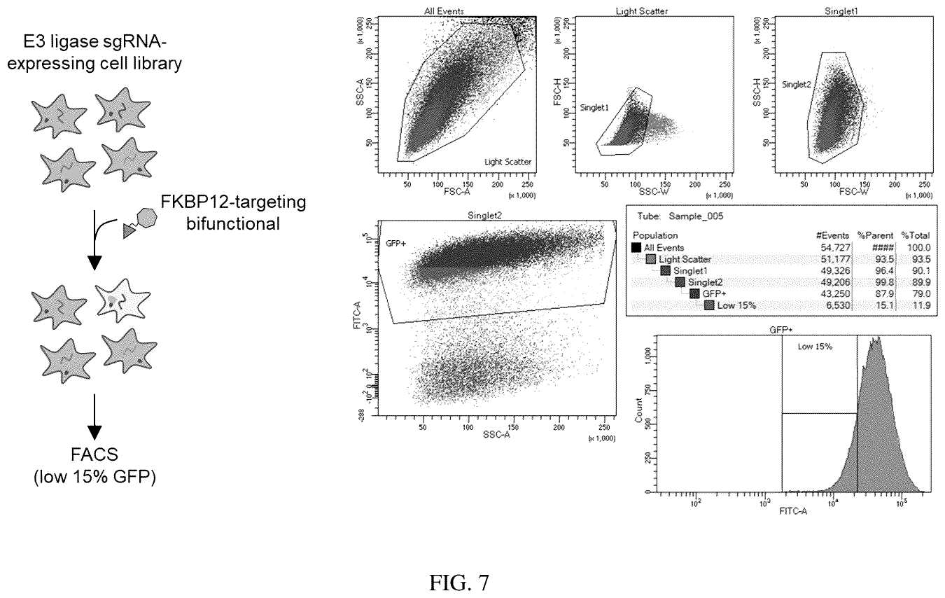

With the CRISPR-Cas9 transcriptional activation cells, a CRISPR-Cas9 transcriptional activation pool screen was developed to identify E3 ligases that support FKBP12 degradation by FKBP12-directed heterobifunctional compounds. To this end, a focused sgRNA library containing 3,520 sgRNAs targeting 680 human E3 ligases (5 sgRNAs per E3 ligase) was generated and packaged into lentivirus (FIG. 1a). The CRISPR-Cas9 transcriptional activation cells were transduced with the pooled sgRNA library at a multiplicity of infection (MOI) of approximately 0.3, yielding approximately 500 cells per sgRNA in each replicate. Following transduction, cells underwent puromycin selection (2 μg/mL) for a duration of 4 days. Subsequent to the selection process, cells were treated with FKBP12-directed candidate PROTACs for 24 hours. Subsequently, cells from the bottom 15% of the GFP population were isolated using fluorescence-activated cell sorting (FIG. 1a and FIG. 7). The relative abundance of sgRNAs in the sorted cells was then compared to the sgRNA distribution in the input cells prior to sorting.

Through an exploration of a series of FKBP12-directed heterobifunctional compounds, a specific candidate, 22-SLF was identified (FIG. 1b), which exhibited substantial enrichment of two distinct sgRNAs targeting the same E3 ligase, FBXO22 (FIG. 1c). This discovery indicates that upon gene expression activation, 22-SLF transforms into a potent degrader, promoting the reduction in FKBP12-EGFP expression. To validate these findings, the two hit sgRNAs targeting FBXO22 promoter regions were introduced into the CRISPR-Cas9 transcriptional activation cells and their capability to activate FBXO22 mRNA expression was confirmed (FIG. 1d). Subsequently, the FBXO22-activated cells were treated with 22-SLF, which resulted in the loss of FKBP12-EGFP expression (FIG. 1e). This data was consistent with the findings from the CRISPR-Cas9 transcriptional activation screen.

### 22-SLF Promotes FBXO22-Dependent Proteasomal Degradation of FKBP12

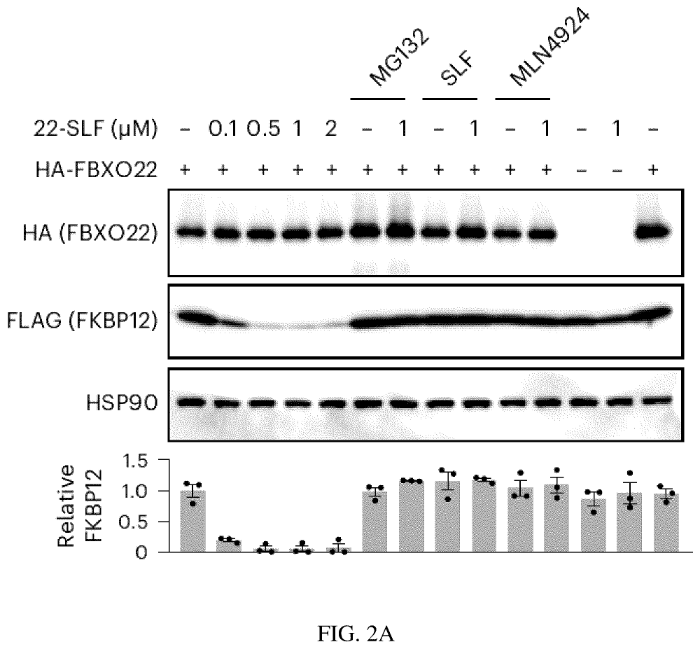

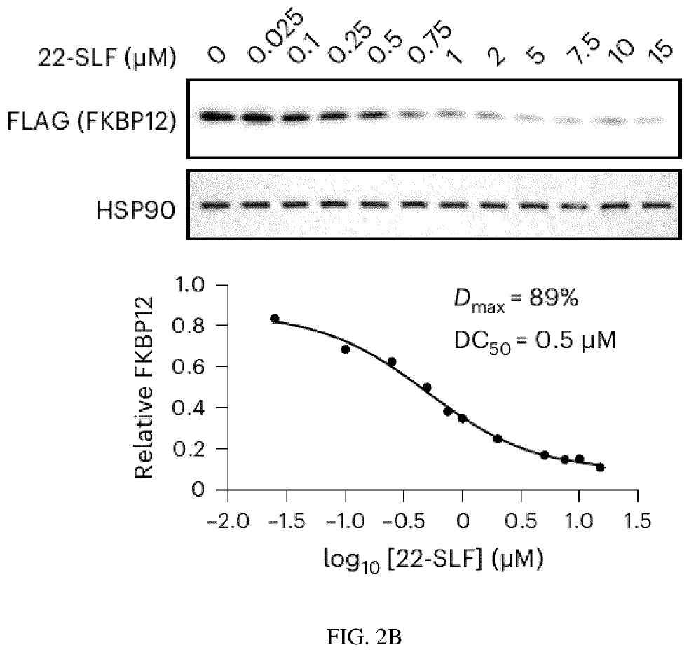

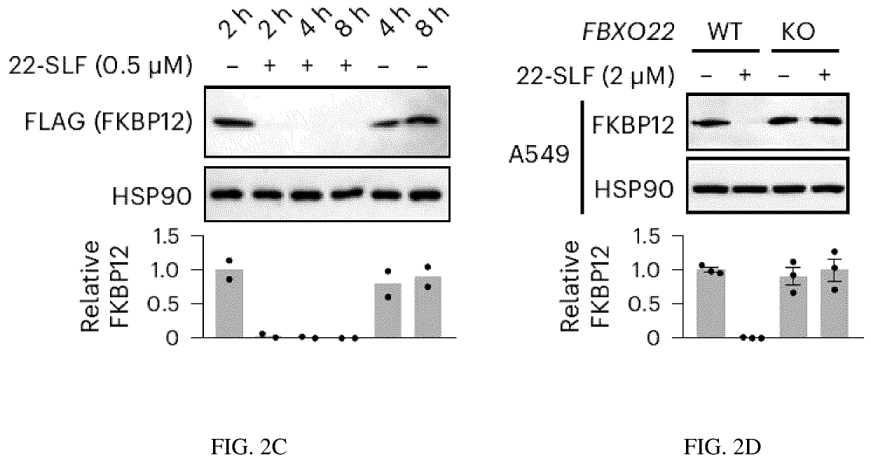

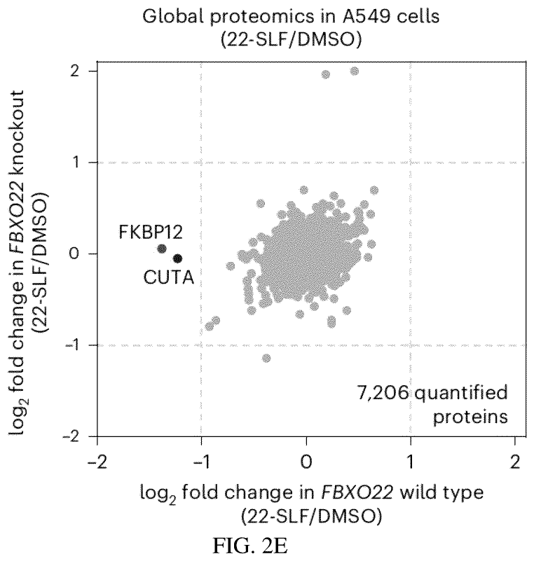

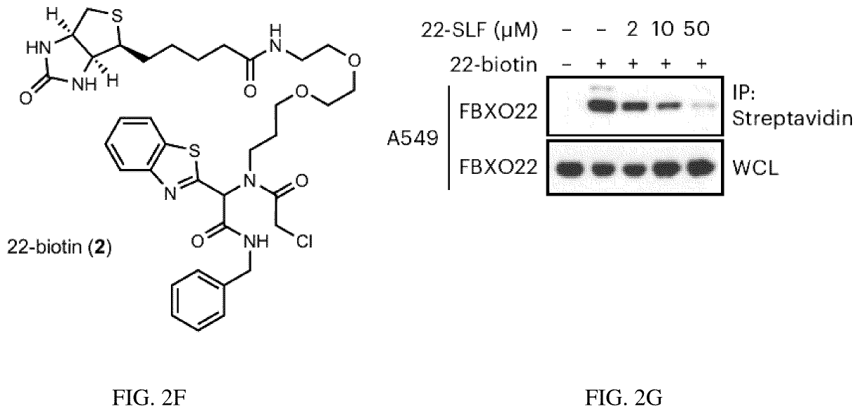

Next, Western blot analysis was used to measure the degradation of FKBP12 induced by 22-SLF. To achieve this, FLAG-tagged FKBP12 and HA-tagged FBXO22 were stably overexpressed in HEK293T cells, followed by subjecting the cells to varying concentrations of 22-SLF. 22-SLF-induced loss of FLAG-FKBP12 expression was FBXO22-dependent, as no reduction was observed in cells lacking HA-FBXO22 (FIG. 2a). Moreover, 22-SLF's effect on FLAG-FKBP12 degradation was concentration-dependent (FIG. 2a). Notably, the loss of FLAG-FKBP12 expression can be blocked by the proteasome inhibitor MG132, as well as the neddylation inhibitor MLN4924 and the FKBP12 ligand SLF (FIG. 2a). The intervention of MG132 in the rescue process indicates that the proteasomal degradation of FLAG-FKBP12 is induced by 22-SLF. The rescue enabled by MLN4924 indicates the involvement of a Cullin-RING ligase (CRL) in the degradation process. This is consistent with FBXO22 belonging to SKP1-CUL1-F-box protein (SCF) ubiquitin ligase complexes, a subfamily of CRL. In addition, the rescue by SLF demonstrates its competitive engagement with the FKBP12 binding site, indicating the role of 22-SLF's binding to FKBP12 for the degradation process. To provide a more comprehensive insight into 22-SLF-induced FKBP12 degradation, a dose-dependent degradation experiment using 11 concentrations of 22-SLF ranging from 0.25 to 15 μM was conducted. This experiment enabled determination of two parameters, the Dmax value, which reached 89%, and the half-maximal degradation concentration (DC50) value, which measured at 0.5 μM (FIG. 2B). Furthermore, the kinetics of 22-SLF-induced degradation of FLAG-FKBP12 are notably rapid, with nearly complete substrate degradation occurring within 2 hours (FIG. 2c). Collectively, this array of data supports the canonical mechanism of ligand-induced protein degradation. In this mechanism, 22-SLF orchestrates the recruitment of FBXO22 to FKBP12, culminating in the proteasomal degradation of FKBP12.

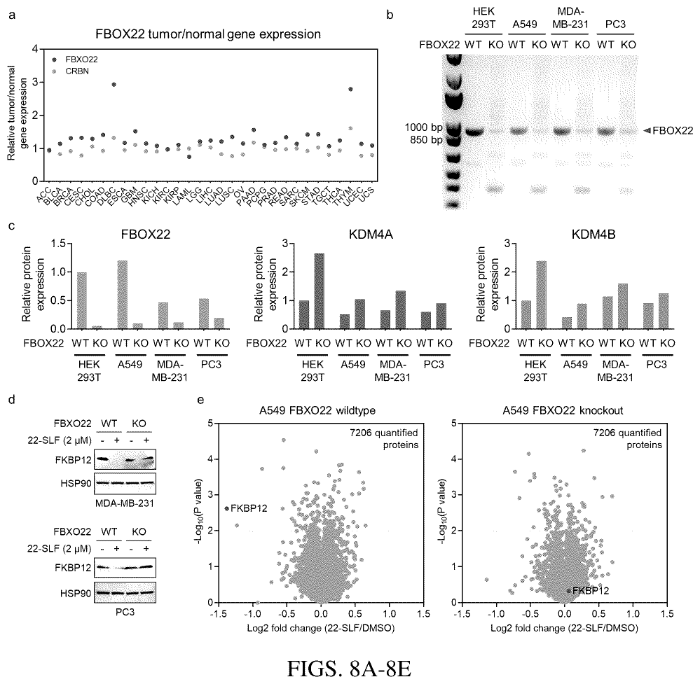

As an F-box protein, FBXO22 is a pivotal player in the context of tumor progression. FBXO22 has many oncogenic functions, including propelling the ubiquitination and subsequent degradation of a diverse spectrum of substrates, such as histone lysine demethylase 4 subfamily KDM4A/B, methylated p53, p21, PTEN, KLF4, and LKB1. Further corroborating its relevance, FBXO22 demonstrates amplified expression within tumor tissues when juxtaposed with normal counterparts (FIG. 8a). To establish the role of endogenous FBXO22 in facilitating 22-SLF-induced FKBP12 degradation, FBXO22 knockout was used in a panel of cancer cell lines, including A549 (lung cancer), MDA-MB-231 (breast cancer), and PC3 (prostate cancer). The efficacy of FBXO22 knockout was verified through both genomic PCR and global proteomic analysis (FIG. 8b,c). Notably, augmented expression of KDM4A and KDM4B—two substrates of FBXO22—was evident in FBXO22 knockout cells, indicating functional disruption of FBXO22 in these cellular contexts (FIG. 8c). Across all three cell lines (A549, MDA-MB-231 and PC3), 22-SLF promoted degradation of endogenous FKBP12, which was abolished upon FBXO22 knockout (FIG. 2d and FIG. 8d). Furthermore, mass spectrometry (MS)-based global proteomic analysis was conducted on A549 parental and FBXO22 knockout cells, following the treatment with 2 μM 22-SLF for 24 hours. This experiment revealed the selective degradation of endogenous FKBP12 in FBXO22 wildtype, but not knockout context (FIG. 2d and FIG. 8e). Another protein, CUTA, was also observed to undergo reduction upon 22-SLF treatment in A549 parental cells, but not in the FBXO22 knockout setting (FIG. 2e and FIG. 8d). To further substantiate the engagement of FBXO22 by the electrophilic portion of 22-SLF, a biotin-conjugated probe known as 22-biotin (FIG. 2f) was synthesized. This probe was then immobilized onto streptavidin-coated beads, which were subsequently subjected to interaction with lysates from HEK293T cells expressing HA-tagged FBXO22. Post-bead washing and on-bead trypsin digestion, the proteins associated with 22-SLF were analyzed using mass spectrometry, leading to the identification of FBXO22 as one of its binding targets (FIG. 2g).

### FBXO22 C227 and C228 are Involved in 22-SLF-Mediated Degradation of FKBP12

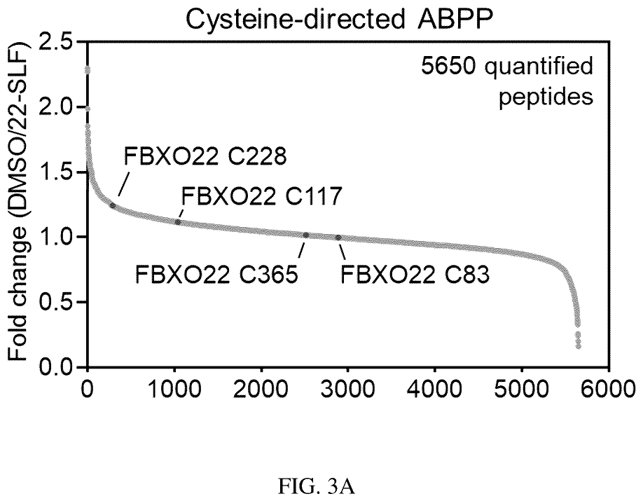

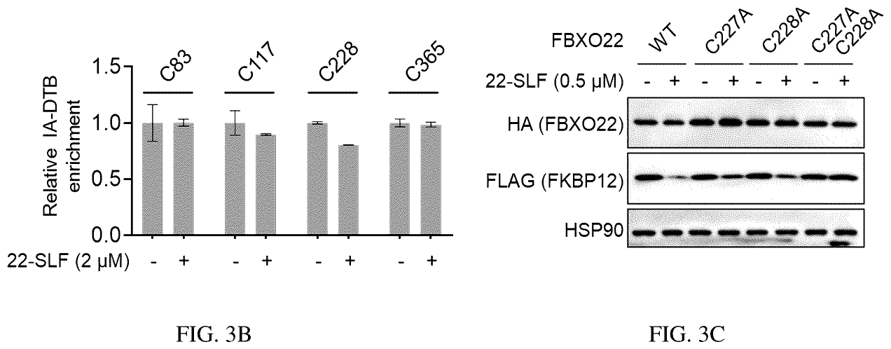

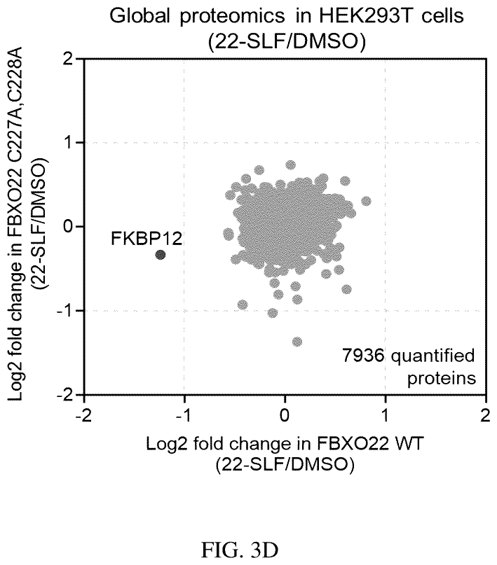

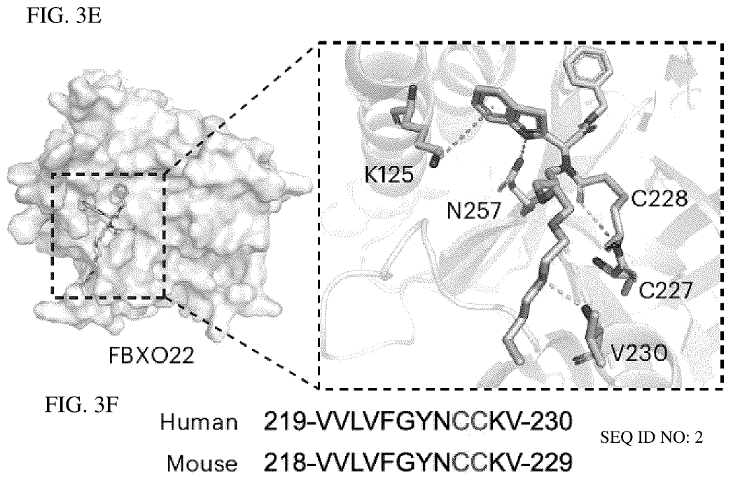

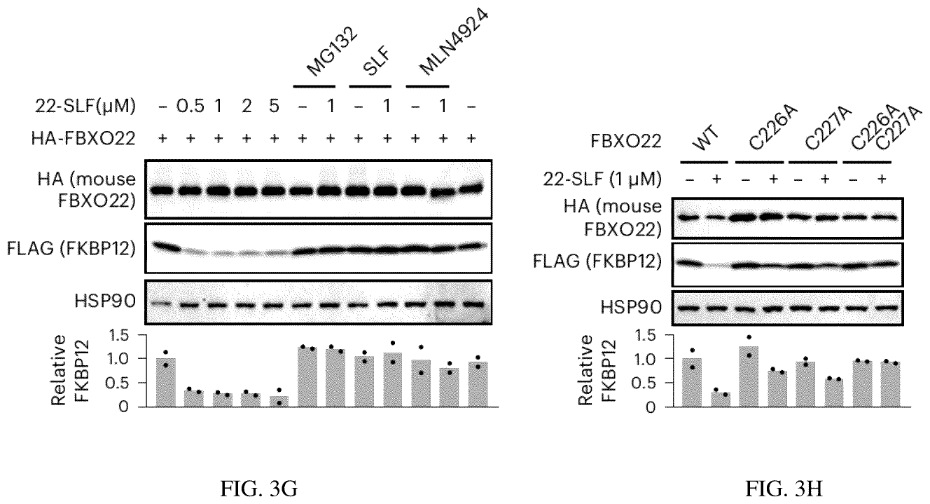

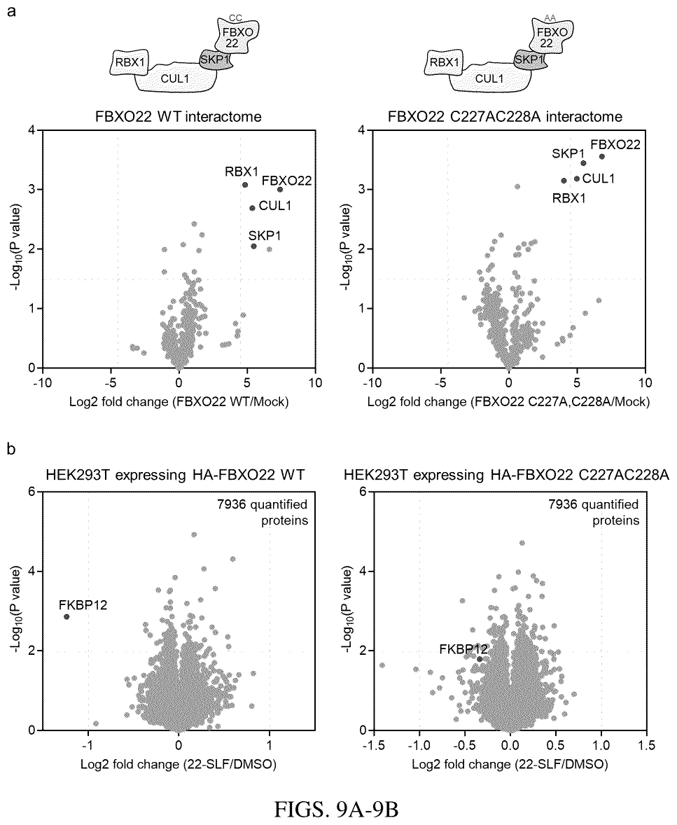

To identify the cysteine residue(s) in FBXO22 engaged with 22-SLF, HA-FBXO22-expressing HEK293T cells were treated with either DMSO or 22-SLF (2 μM, 2 hours). Using competitive cysteine-directed activity-based protein profiling (ABPP), a chemical proteomics for the measurement of target engagement, the degree of blockade of iodoacetamide-desthiobiotin (IA-DTB)-modified cysteines on FBXO22 was quantified. From a pool of 5,650 quantified IA-DTB-modified peptides, four cysteines on FBXO22 (C83, C117, C228, and C365) (FIG. 3a) were identified, with approximately 20% of FBXO22 C228 being blocked by 22-SLF treatment (FIG. 3b), indicating the potential interaction with this cysteine by 22-SLF. Consequently, C228 was mutated to alanine and HA-FBXO22 C228A-expressing HEK293T cells were generated (FIG. 3c). Interestingly, mutating C228 to alanine did not fully abolish 22-SLF-induced degradation of FKBP12, as the compound still partially degraded FKBP12 (FIG. 3c). Notably, in close proximity to C228 lies another cysteine, C227, indicating the potential redundancy of these two cysteines in facilitating 22-SLF-induced FKBP12 degradation. Furthermore, the similarity in the fragmentation pattern of C227- and C228-modified peptides during MS analysis indicates that the engagement of C227 in FBXO22 by 22-SLF might not have been captured in the ABPP analysis. Another single mutant (C227A) and a double mutant (C227AC228A) were generated in HEK293T cells expressing these variants and 22-SLF-induced FKBP12 degradation was assessed. The data revealed that both single mutants, C227A and C228A, partially impeded 22-SLF-induced FKBP12 degradation, and the double mutant, C227AC228A, completely blocked the degradation of FKBP12 induced by 22-SLF (FIG. 3c). This demonstrates that C227 and C228 have overlapping roles in supporting 22-SLF-mediated FKBP12 degradation. To address the concern regarding potential conformational changes caused by mutations affecting the degradation, affinity purification-mass spectrometry (AP-MS) comparing the interactome of FBXO22 wildtype and C227AC228A double mutant was conducted. The results indicated that both forms were incorporated into the SKP1-CUL1-RBX1 E3 complex (FIG. 9a), indicating proper folding of FBXO22 C227AC228A double mutant.

Global proteomic analysis in FBXO22 wildtype- and C227AC228A-expressing HEK293T cells was conducted, comparing the effects of 22-SLF treatment on the expression level of FKBP12. The findings revealed that among 7,936 quantified proteins, FKBP12 was the only protein degraded by 22-SLF in FBXO22 wildtype expressing cells, with no degradation observed in FBXO22 C227AC228A expressing cells (FIG. 3d and FIG. 9b). Although the three-dimensional structure of FBXO22 remains undetermined, AlphaFold predictions demonstrate high confidence (pLDDT>90) across most of the protein sequence, including the peptide encompassing liganded C227 and C228. Using this predicted human FBXO22 structure, a model structure of the interaction with the electrophilic portion of 22-SLF was conducted. The model indicates the formation of multiple hydrogen bonds between the ligand and a pocket on FBXO22 (FIG. 3e).

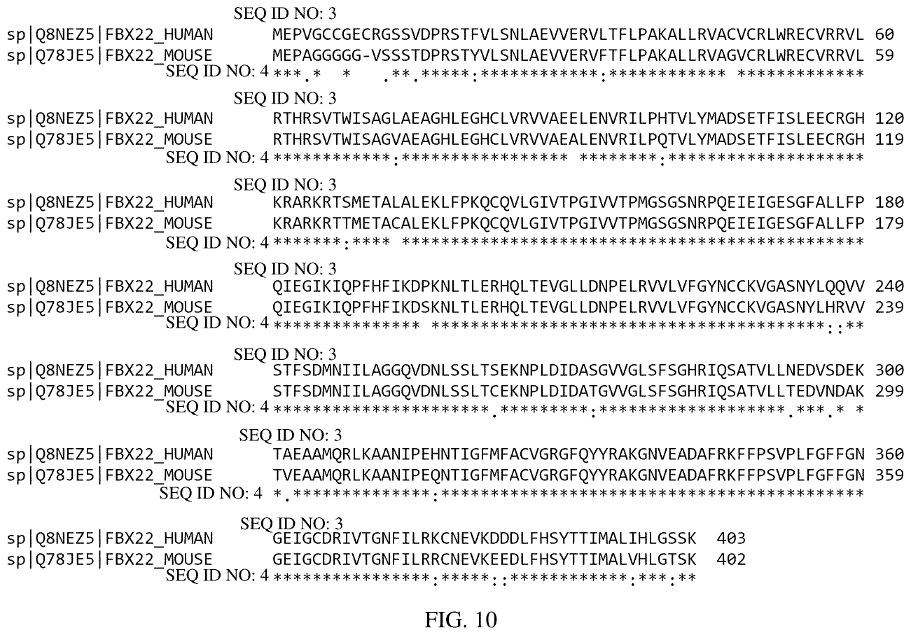

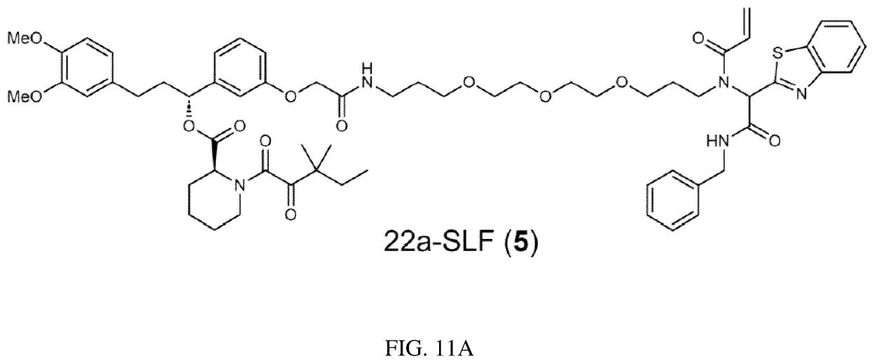

It's noteworthy that FBXO22, a 403-amino acid protein, exhibits remarkable conservation across mammals, with human and mouse FBXO22 sharing 93% identity (FIG. 10). Intriguingly, the peptide sequence containing the two consecutive cysteines (C227 and C228) is identical between human and mouse (FIG. 3f). To explore whether mouse FBXO22 can facilitate 22-SLF-induced FKBP12 degradation and if the conserved cysteines (C226 and C227 in mouse FBXO22) are pivotal, HEK293T cells expressing mouse FBXO22 wildtype, C226A, C227A, and C226AC227A, were generated. The results showed that 22-SLF treatment induced FBKP12 degradation in mouse FBXO22 wildtype-expressing cells, which can be blocked by MG132, SLF, and MLN4924 (FIG. 3g,h). Similar to human FBXO22, the single mutants, C226A and C227A, partially impeded degradation, while the double mutant, C226AC227A, fully blocked the degradation (FIG. 3g). This data underlines that mouse FBXO22 can integrate effectively into the human SKP1-CUL1-RBX1 complex and function as an E3 ligase supporting 22-SLF-induced FKBP12 degradation.

### 22-SLF Induces a Ternary Complex Between FKBP12 and FBXO22

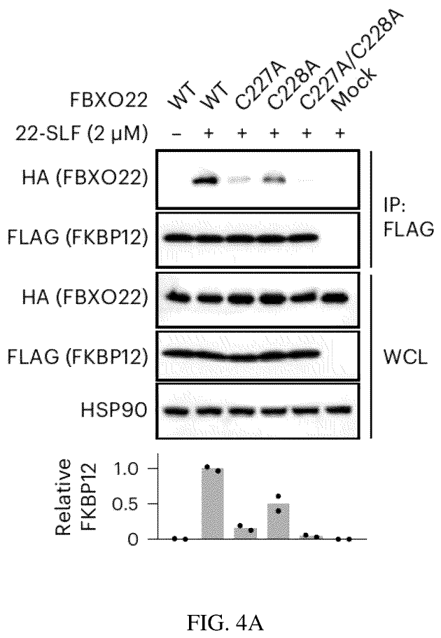

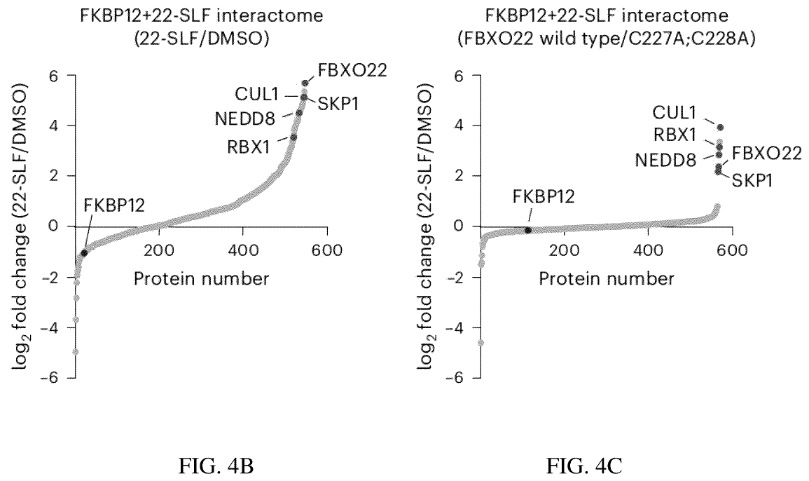

The formation of ternary complex by degraders with their target protein and E3 ligase drives the subsequent target degradation. To demonstrate the ternary complex involving 22-SLF, FKBP12 and FBXO22, HEK293T cells expressing HA-FBXO22 and FLAG-FKBP12 were treated with 22-SLF and a proteasomal inhibitor MG132. This experiment reveals that HA-FBXO22 co-immunoprecipitated with FLAG-FKBP12 in the presence of 22-SLF and MG132 (FIG. 4a), supporting a ternary complex formation involving 22-SLF, FBXO22, and FKBP12. Notably, the assembly of this ternary complex was partially hindered upon expression of FBXO22 C227A or C228A single mutant, and nearly completely abolished when the FBXO22 C227AC228A double mutant was introduced (FIG. 4a). This data further demonstrates that 22-SLF engages C227/228 in FBXO22, leading to the formation of a ternary complex with and subsequent degradation of FKBP12.

To comprehensively assess the impact of 22-SLF on the FKBP12 interactome landscape, an enrichment proteomic approach was employed to identify proteins co-immunoprecipitating with FLAG-FKBP12 from HEK293T cells treated with 22-SLF. This analysis revealed several protein components of the FBXO22 complex, including FBXO22 itself, SKP1, CUL1, and NEDD8, as proteins recruited by 22-SLF (FIG. 4b). Furthermore, another enrichment proteomic study was conducted comparing FKBP12 interactome landscape in HEK293T cells expressing FBXO22 wildtype and C227AC228A double mutant. This experiment revealed the enrichment of the FBXO22 complex in FBXO22 wildtype-expressing cells, whereas the enrichment was absent in FBXO22 C227AC228A-expressing cells (FIG. 4c). Collectively, these findings indicate that 22-SLF effectively drives the formation of a ternary complex involving 22-SLF, FBXO22 and FKBP12 by engaging C227/228 in FBXO22, thereby facilitating the subsequent degradation of FKBP12.

### Harnessing FBXO22 for the Degradation of Additional Protein Targets

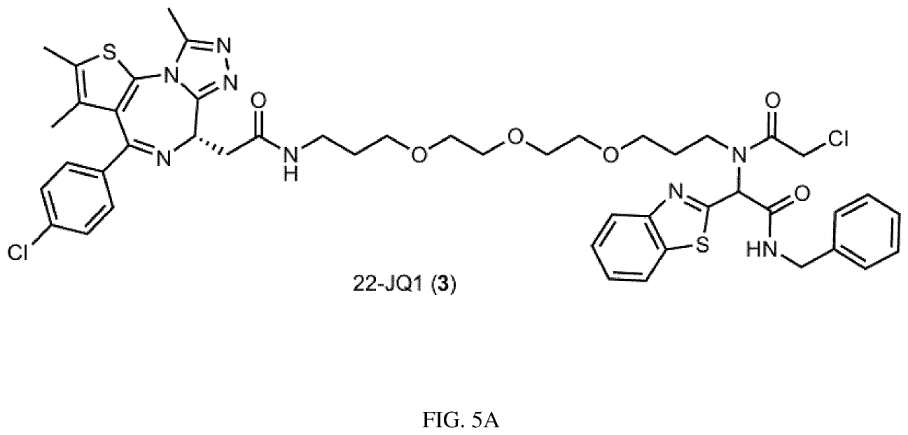

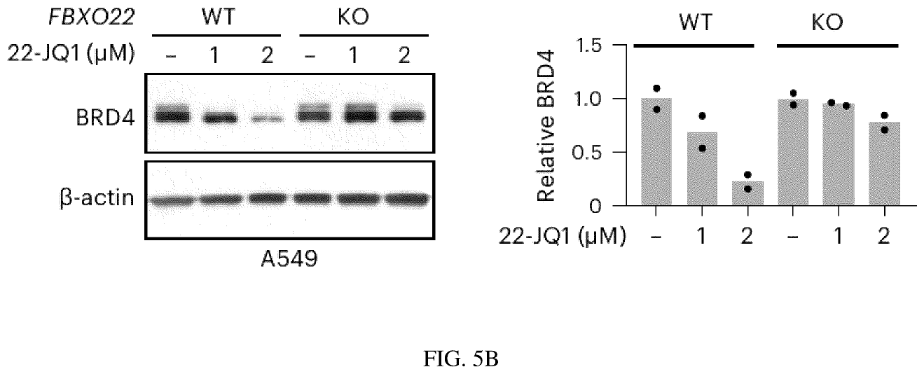

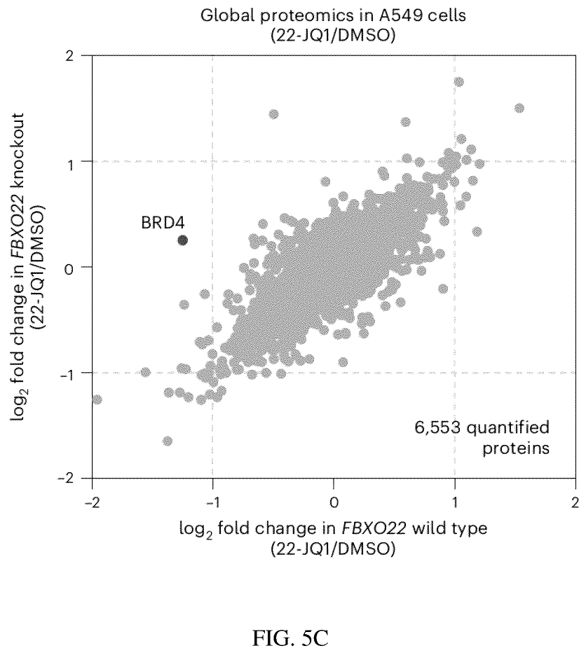

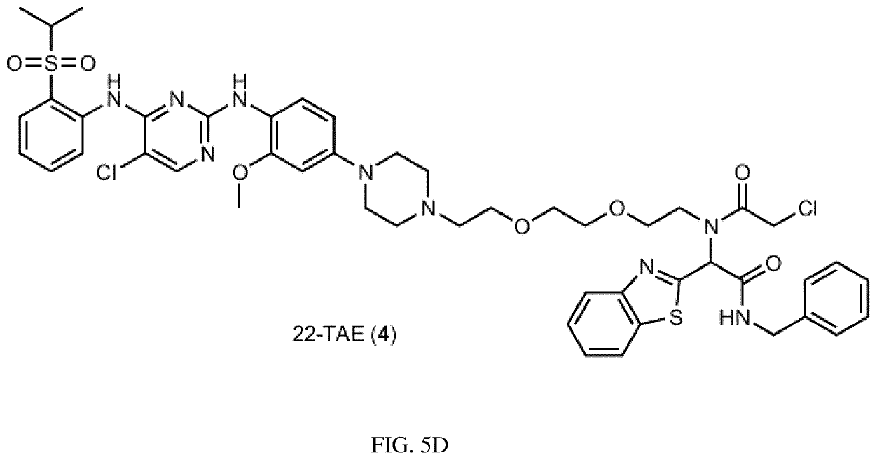

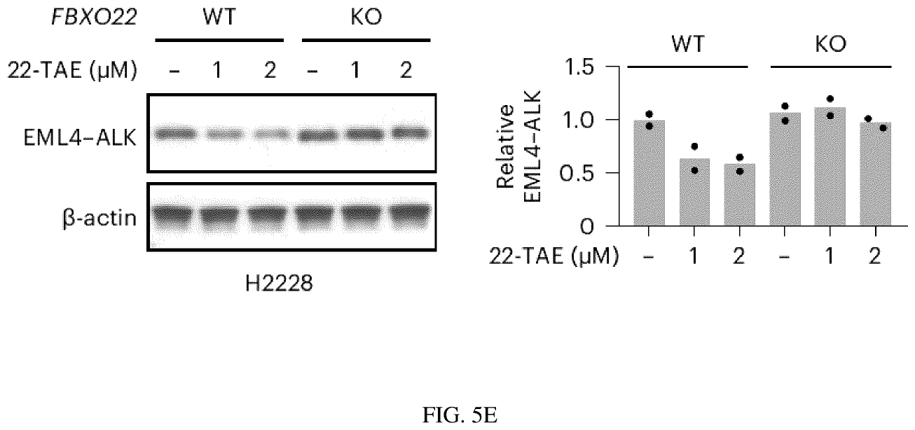

Next, the potential of FBXO22 to facilitate the degradation of additional proteins was investigated. Bromodomain-containing protein 4 (BRD4) was chosen due to its notable physiological role and the availability of a selective ligand, JQ1. The compound, 22-JQ1 (3) was synthesized by coupling the FBXO22 binding moiety to JQ1 (FIG. 5a). 22-JQ1 promoted the degradation of BRD4 in A549 wild-type cells, which was abolished upon FBXO22 knockout (FIG. 5b). Subsequently, a global proteomic analysis was performed in A549 wild-type and FBXO22-knockout cells treated with 22-JQ1. 22-JQ1 induced a potent degradation of BRD4 in A549 wild-type but not FBXO22-knockout cells (FIG. 5c). To further demonstrate the potential of using FBXO22 for degrading additional targets, another PROTAC, 22-TAE (4) was synthesized. 22-TAE incorporates the FBXO22 binding moiety coupled with a defined anaplastic lymphoma kinase (ALK) ligand (FIG. 5d). FBXO22 knockout was generated in H2228, a non-small cell lung cancer cell line expressing the echinoderm microtubule-associated protein-like 4 (EML4)-ALK fusion protein. 22-TAE induced the degradation of EML4-ALK fusion protein in H2228 wild-type cells, while no such degradation was observed in FBXO22-knockout H2228 cells (FIG. 5e). Taken together, these results underscore the potential of FBXO22 in the creation of PROTACs capable of degrading multiple protein targets.

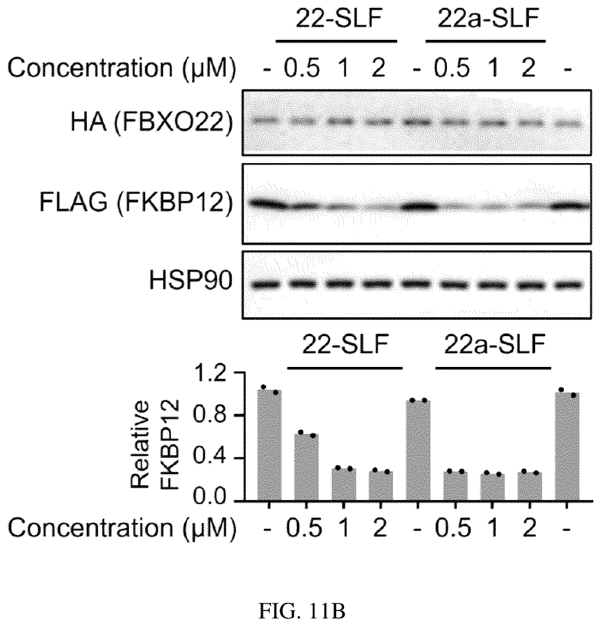

Lastly, considering that α-chloroacetamide is a highly reactive electrophilic warhead, there may be challenges in advancing to the therapeutic stage because of its potential engagement of off-target cysteines, such as catalytic cysteines. To this end, an acrylamide variant of 22-SLF, 22a-SLF (5), was synthesized (FIG. 11A). Intriguingly, 22a-SLF facilitated the degradation of FKBP12 with similar potency to 22-SLF (FIG. 11B). These results suggest that embarking on a future medicinal chemistry campaign using the acrylamide warhead could be a promising strategy to pursue.

In this study, a CRISPR-Cas9 transcriptional activation screen was developed and used to unveil the capacity of the E3 ligase FBXO22 in supporting targeted protein degradation when engaged by electrophilic PROTACs. This novel approach holds promise for broad applications, enabling the exploration of heterobifunctional compounds for their potential to degrade a diverse array of proteins of interest. Given the distinct gene expression profiles of various cell lines, candidate heterobifunctional compounds might exhibit an “inactive” phenotype due to the absence of compatible E3 ligases. The method developed herein offers an impartial means of significantly boosting E3 ligase expression, facilitating confident assessment of candidate heterobifunctional compounds. This approach is adaptable to both covalent and non-covalent compounds.

In instances where candidate compounds display moderate activity leading to partial target protein degradation, CRISPR-Cas9 knockout screens can yield high background noise, obscuring the deconvolution of specific E3 ligases. By activating E3 ligases, as performed herein, more pronounced target degradation is observed, allowing for the enrichment of sgRNAs associated with the relevant E3 genes. Additionally, this technique holds value in cases where certain protein degraders exhibit degradation solely in specific cell lines or primary cells, which can pose challenges for establishing CRISPR-Cas9 knockout screens in hard-to-transduce cell models. In these situations, a CRISPR-Cas9 transcriptional activation screen could be established in transducible cells such as HEK293T cells. Absent activation of E3 ligase genes, the compound would exhibit inactive degradation behavior, but upon activating the target E3 ligase gene, the compound would become active, facilitating the isolation of cells where target degradation has occurred. Furthermore, this CRISPR-Cas9 transcriptional activation screen approach can serve to uncover novel endogenous degradation pathways, as well as identify redundant degradation pathways. By globally activating the expression of the entire E3 ligase family, the capabilities of all E3 ligases in degrading proteins of interest can be evaluated.

FBXO22 is an E3 ligase involved in tumor progression. Given its elevated expression in tumors compared to normal tissue (FIG. 8a), FBXO22 could offer an advantageous avenue for the degradation of cancer targets with potentially minimized toxicity in normal cells. Despite the extensive research into FBXO22's biology, there remains a scarcity of studies focused on developing inhibitors or ligands for FBXO22. The investigation herein establishes a foundational step in the development of FBXO22-targeting ligands with therapeutic potential and provides novel PROTACS 22-SLF, 22a-SLF, 22-TAE, and 22-JQ1. For example, the present study demonstrates that approximately 20% engagement of FBXO22 C227 by 22-SLF effectively induces the degradation of FKBP12 (FIG. 3b), indicating that electrophilic PROTACs could achieve efficient degradation of target proteins with only partial engagement of E3 ligases, maintaining the physiological function of these ligases. Conversely, given the pivotal role of FBXO22 in tumor progression, inhibiting FBXO22 could hold promise as a cancer treatment strategy. Thus, for cell contexts where FBXO22 inhibition could confer therapeutic benefits, potent ligands exhibiting high engagement with FBXO22 for degrader development could yield dual pharmacological effects—degradation of target proteins alongside FBXO22 inhibition.

What is claimed is:

1. A proteolysis-targeting chimera (PROTAC) comprising:
a) a domain that binds to E3 ligase F-box protein 22 (FBXO22),
b) a target-binding domain, and
c) a linker connecting the domain that binds to FBXO22 to the target-binding domain, wherein the domain that binds to FBXO22 comprises the structure:

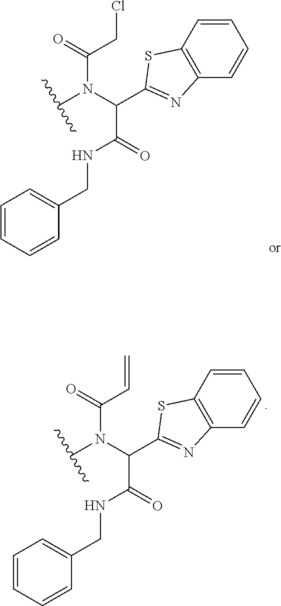 [SMILES: *N(C(=O)CCl)C(C(=O)NCc1ccccc1)c1nc2ccccc2s1 ; *N(C(=O)C=C)C(C(=O)NCc1ccccc1)c1nc2ccccc2s1]

2. The PROTAC of claim 1, wherein the linker comprises one or more alkylene oxide units.

3. The PROTAC of claim 1, wherein binding of the PROTAC to FBXO22 and to the target induces FBXO22-mediated targeted protein degradation of the target.

4. The PROTAC of claim 1, wherein the target is 12-kDa FK506-binding protein (FKBP12).

5. The PROTAC of claim 4, wherein the target-binding domain comprises the structure:

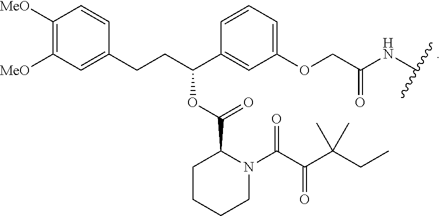 [SMILES: *NC(=O)COc1cccc([C@@H](CCc2ccc(OC)c(OC)c2)OC(=O)[C@@H]2CCCCN2C(=O)C(=O)C(C)(C)CC)c1]

6. The PROTAC of claim 5, wherein the PROTAC is 22-SLF, defined by the structure:

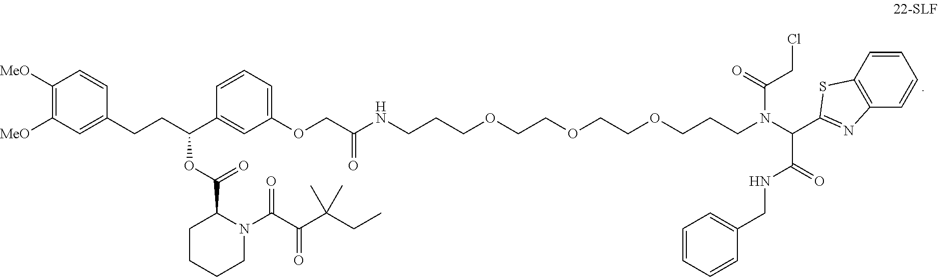 [SMILES: CCC(C)(C)C(=O)C(=O)N1CCCC[C@H]1C(=O)O[C@H](CCc1ccc(OC)c(OC)c1)c1cccc(OCC(=O)NCCCOCCOCCOCCCN(C(=O)CCl)C(C(=O)NCc2ccccc2)c2nc3ccccc3s2)c1]

7. The PROTAC of claim 5, wherein the PROTAC is 22-aSLF, defined by the structure:

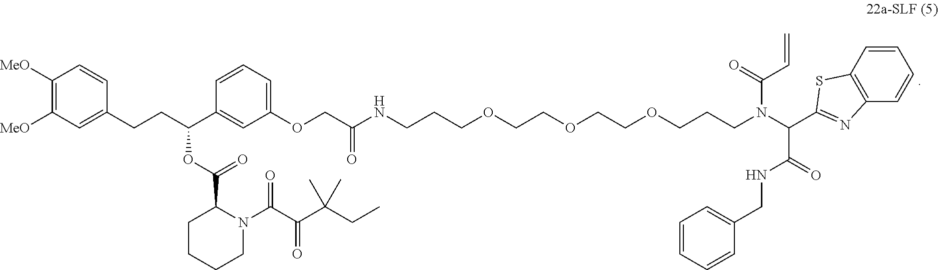 [SMILES: C=CC(=O)N(CCCOCCOCCOCCCNC(=O)COc1cccc([C@@H](CCc2ccc(OC)c(OC)c2)OC(=O)[C@@H]2CCCCN2C(=O)C(=O)C(C)(C)CC)c1)C(C(=O)NCc1ccccc1)c1nc2ccccc2s1]

8. The PROTAC of claim 1, wherein the target is bromodomain-containing protein 4 (BRD4).

9. The PROTAC of claim 8, wherein the target-binding domain comprises the structure:

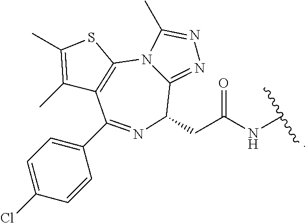 [SMILES: *NC(=O)C[C@@H]1N=C(c2ccc(Cl)cc2)c2c(sc(C)c2C)-n2c(C)nnc21]

10. The PROTAC of claim 9, wherein the PROTAC is 22-JQ1, defined by the structure:

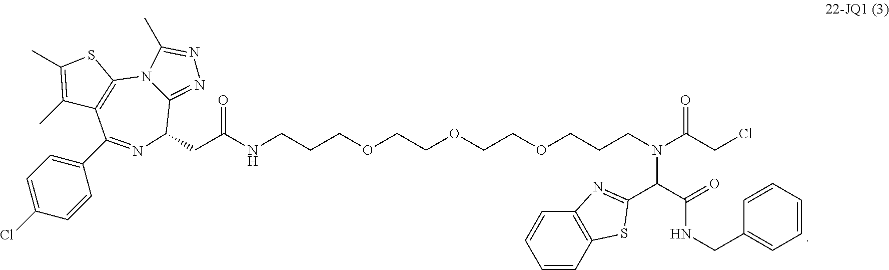 [SMILES: Cc1sc2c(c1C)C(c1ccc(Cl)cc1)=N[C@@H](CC(=O)NCCCOCCOCCOCCCN(C(=O)CCl)C(C(=O)NCc1ccccc1)c1nc3ccccc3s1)c1nnc(C)n1-2]

11. The PROTAC of claim 1, wherein the target is anaplastic lymphoma kinase (ALK).

12. The PROTAC of claim 11, wherein the target-binding domain comprises the structure:

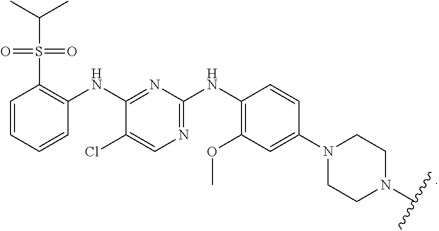 [SMILES: *N1CCN(c2ccc(Nc3ncc(Cl)c(Nc4ccccc4S(=O)(=O)C(C)C)n3)c(OC)c2)CC1]

13. The PROTAC of claim 12, wherein the PROTAC is 22-TAE, defined by the structure:

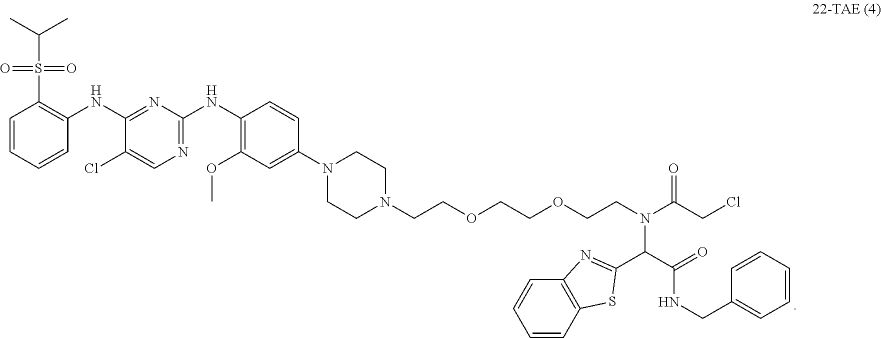 [SMILES: COc1cc(N2CCN(CCOCCOCCN(C(=O)CCl)C(C(=O)NCc3ccccc3)c3nc4ccccc4s3)CC2)ccc1Nc1ncc(Cl)c(Nc2ccccc2S(=O)(=O)C(C)C)n1]

14. A method of promoting targeted protein degradation in a cell or a subject, or a method of treating a neurodegenerative disease or cancer in a subject, the method comprising administering to the cell or subject the PROTAC of claim 1.

15. The method of claim 14, wherein the neurodegenerative disease is Alzheimer's disease or Parkinson's disease.

16. A method of identifying E3 ligases that support targeted protein degradation (TPD), the method comprising:
a) providing a sample comprising cells expressing a fusion protein and a CRISPR-Cas9 transcriptional activation system, wherein the fusion protein comprises a target protein and a reporter molecule;
b) transducing the cells with an sgRNA library targeting human E3 ligases;
c) treating the cells with an agent putatively directed against the target protein, wherein a reduction of signal from the reporter molecule indicates targeted protein degradation of the target protein;
d) isolating cells with a reduced signal from the reporter molecule; and
e) determining the expression of sgRNAs in the isolated cells relative to the expression of sgRNAs in the sgRNA library.

17. The method of claim 16, further comprising identifying one or more E3 ligases targeted by sgRNAs having enriched expression in the isolated cells, wherein the E3 ligases identified are determined to support targeted protein degradation of the target protein.

18. The method of claim 16, wherein the sgRNA library comprises at least 1,000 sgRNAs.

19. A method of identifying whether an agent induces targeted protein degradation (TPD) of a target protein, the method comprising:
a) providing a sample comprising cells expressing a fusion protein and a CRISPR-Cas9 transcriptional activation system, wherein the fusion protein comprises a target protein and a reporter molecule;
b) transducing the cells with an sgRNA library targeting human E3 ligases;
c) treating the cells with the agent; and
d) evaluating a signal from the reporter molecule in one or more cells, wherein a reduction of signal from the reporter molecule indicates that the agent induces TPD of the target protein.

20. The method of claim 19, wherein the sgRNA library comprises at least 1,000 sgRNAs.


SEQUENCE LISTING (4 sequences)

SEQ ID NO: 1 - protein, 403 aa, synthetic construct
MEPVGCCGECRGSSVDPRSTFVLSNLAEVVERVLTFLPAKALLRVACVCRLWRECVRRVLRTHRSVTWISAGLAEAGHLEGHCLVRVVAEELENVRILPHTVLYMADSETFISLEECRGHKRARKRTSMETALALEKLFPKQCQVLGIVTPGIVVTPMGSGSNRPQEIEIGESGFALLFPQIEGIKIQPFHFIKDPKNLTLERHQLTEVGLLDNPELRVVLVFGYNCCKVGASNYLQQVVSTFSDMNIILAGGQVDNLSSLTSEKNPLDIDASGVVGLSFSGHRIQSATVLLNEDVSDEKTAEAAMQRLKAANIPEHNTIGFMFACVGRGFQYYRAKGNVEADAFRKFFPSVPLFGFFGNGEIGCDRIVTGNFILRKCNEVKDDDLFHSYTTIMALIHLGSSK

SEQ ID NO: 2 - protein, 12 aa, synthetic construct
VVLVFGYNCCKV

SEQ ID NO: 3 - protein, 399 aa, synthetic construct
MEPVGCCGECRGSSVDPRSTFVLSNLAEWERVLTFLPAKALLRVACVCRLWRECVRRVLRTHRSVTWISAGLAEAGHLEGHCLVRWAEELENVRILPHTVLYMADSETFISLEECRGHKRARKRTSMETALALEKLFPKQCQVLGIVTPGIWTPMGSGSNRPQEIEIGESGFALLFPQIEGIKIQPFHFIKDPKNLTLERHQLTEVGLLDNPELRVVLVFGYNCCKVGASNYLQQVVSTFSDMNIILAGGQVDNLSSLTSEKNPLDIDASGWGLSFSGHRIQSATVLLNEDVSDEKTAEAAMQRLKAANIPEHNTIGFMFACVGRGFQYYRAKGNVEADAFRKFFPSVPLFGFFGNGEIGCDRIVTGNFILRKCNEVKDDDLFHSYTTIMALIHLGSSK

SEQ ID NO: 4 - protein, 398 aa, synthetic construct
MEPAGGGGGVSSSTDPRSTYVLSNLAEWERVFTFLPAKALLRVAGVCRLWRECVRRVLRTHRSVTWISAGVAEAGHLEGHCLVRWAEALENVRILPQTVLYMADSETFISLEECRGHKRARKRTTMETACALEKLFPKQCQVLGIVTPGIWTPMGSGSNRPQEIEIGESGFALLFPQIEGIKIQPFHFIKDSKNLTLERHQLTEVGLLDNPELRVVLVFGYNCCKVGASNYLHRVVSTFSDMNIILAGGQVDNLSSLTCEKNPLDIDATGWGLSFSGHRIQSATVLLTEDVNDAKTVEAAMQRLKAANIPEQNTIGFMFACVGRGFQYYRAKGNVEADAFRKFFPSVPLFGFFGNGEIGCDRIVTGNFILRRCNEVKEEDLFHSYTTIMALVHLGTSK